(19) United States
(12) Patent Application Publication
(10) Pub. No.: US 2016/0096819 A1
(43) Pub. Date: Apr. 7, 2016

(54) PROCESS FOR PREPARING N-SUBSTITUTED 1H-PYRAZOLE-5-CARBOXYLIC ACID COMPOUNDS AND DERIVATIVES THEREOF

(71) Applicant: BASF SE, Ludwigshafen (DE)

(72) Inventors: Christopher KORADIN, Gommersheim (DE); Thomas ZIERKE, Boehl-Iggelheim (DE); Ralf HOOCK, Ludwigshafen (DE); Karsten KOERBER, Eppelheim (DE)

(21) Appl. No.: 14/890,711

(22) PCT Filed: May 16, 2014
(86) PCT No.: PCT/EP2014/060082

§ 371 Date: Nov. 12, 2015

(30) Foreign Application Priority Data
May 17, 2013 (EP) 13168365.8

(51) Int. Cl. C07D 401/04

(52) U.S. Cl. CPC C07D 401/04

(57) ABSTRACT

The present invention relates to a process for preparing N-substituted 1H-pyrazole-5-carboxylic acid compounds of the formula I-A and derivatives thereof, in particular the corresponding carbonylchloride compounds (acid chlorides). It also relates to the use of these acid chlorides for preparing anthranilamide derivatives that are useful pesticides. in which the variables are as defined in the claims and the specification comprising the steps of i) reacting a compound of the formula (II) with a base selected from combinations of a magnesium-organic compound having a carbon bound magnesium and a secondary amine and magnesium amides of secondary amines in the presence of a lithium halide, where the base is used in an amount sufficient to achieve at least 80% deprotonation of the compound of formula (II); and subjecting the product obtained in step (i) to a carboxylation by reacting it with carbon dioxide or a carbon dioxide equivalent, to obtain a magnesium salt of the compound of formula (I-A) and optionally aqueous workup to obtain the compound of the formula (I-A) as a free acid.

The present invention relates to a process for preparing N-substituted 1H-pyrazole-5-carboxylic acid compounds and derivatives thereof, in particular the corresponding carbonylchloride compounds (acid chlorides). It also relates to the use of these acid chlorides for preparing anthranilamide derivatives that are useful pesticides.

N-substituted 1H-pyrazole-5-carboxylate compounds and the corresponding acid chlorides, in particular substituted 1-pyridin-2-yl-1H-pyrazole-5-carbonylchlorides are important precursors for anthranilamide derivates that carry a 1-pyridin-2-yl-1H-pyrazol-5-yl-carbonyl substituent at the aromatic amino group. Such compounds find use as pesticides, especially as insecticides, which are disclosed, for example, in WO 01/70671, WO 03/015518, WO 03/015519, WO 03/016284, WO 03/016300, WO 03/024222, WO 06/000336; WO 06/068669, WO 07/043677 and WO 08/130021.

For preparation of substituted 1-pyridin-2-yl-1H-pyrazole-5-carbonylchlorides, a process described in WO 02/070483, WO03/015519, WO 07/043677 and WO 08/130021 has been found to be useful. It is based on the deprotonation of a 1-pyridin-2-yl-1H-pyrazole compound with either n-butyl lithium or lithium diisopropylamide, followed by reacting the resulting lithiated species with carbon dioxide to the corresponding carboxylic acid, which is subsequently chlorinated using a dehydrative chlorinating agent such as thionyl chloride or oxalyl chloride to give the corresponding acid chloride. Similar synthetic routes that all require the formation of the pyrazole-5-carboxylic acid as an intermediate are described for example in: Khimiya Geterotsiklicheskikh Soedinenii 1975, 3, 392-395; Heterocycles 1985, 23, 943-951; Bioorganic & Medicinal Chemistry Letters 2005, 15, 4898-4906; WO 06/000336; WO 06/068669; Bioorganic & Medicinal Chemistry Letters 2007, 17, 6274-6279; Bioorganic & Medicinal Chemistry 2008, 16, 3163-3170; Organic Reactions 1979, 26; Bioorganic & Medicinal Chemistry Letters 2008, 18, 4438-4441 and WO 08/011131.

However, these procedures of the prior art suffer from several limitations rendering them hardly suitable for industrial scale production. For instance, the application of the highly reactive organolithium bases, such as methyllithium butyllithium, phenyllithium or lithium diisopropylamide, for the deprotonation of pyrazoles represents a potentially hazardous step in the synthesis, in particular if performed on a large scale. Moreover, these organolithium bases are very expensive and require very low reaction temperatures, which in itself already results in excessive energy costs. Additionally, a conversion of 1-pyridin-2-yl-1H-pyrazole compounds to the corresponding pyrazole-5-carboxylic acid chloride in less than the four steps required by the known procedures, would be highly desirable, as every synthetic step is time and energy consuming and leads to a loss of material.

It is an object of the present invention to provide processes for preparing N-substituted 1H-pyrazole-5-carboxylate and N-substituted 1H-pyrazole-5-carbonylchloride compounds and for preparing pyrazolecarboxamides of anthranilamides derived therefrom. These processes should be simple to carry out, require 4 or 3 or less steps and be suitable for the industrial scale production. They should additionally be inexpensive and safe, be based on selective reactions and afford the desired product in a short reaction time using moderate reaction conditions.

The object is achieved by the processes described in detail hereinafter.

A first aspect of the present invention relates to a process for preparing an N-substituted 1H-pyrazole-5-carboxylic acid of the formula (I-A) or a magnesium salt thereof

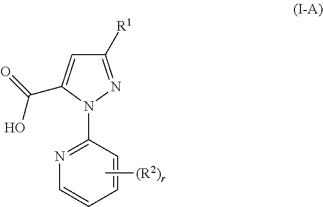

in which

R¹ is selected from hydrogen, halogen, cyano, —SF₅, C₁-C₆-alkyl, C₁-C₆-fluoroalkyl, CBrF₂, C₃-C₈-cycloalkyl, C₃-C₈-fluorocycloalkyl, C₂-C₆-alkenyl, C₂-C₆-fluoroalkenyl, wherein the six last mentioned radicals may be substituted by one or more radicals R^{a}; —Si(R^{f})₂R^{g}, —OR^{b}, —SR^{b}, —S(O)ₘR^{b}, —S(O)ₙN(R^{b})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R^{e};

each R² is independently selected from the group consisting of halogen, SF₅, C₁-C₆-alkyl, C₁-C₆-fluoroalkyl, C₃-C₈-cycloalkyl, C₃-C₈-fluorocycloalkyl, C₂-C₆-alkenyl, C₂-C₆-fluoroalkenyl, wherein the six last mentioned radicals may be substituted by one or more radicals R^{a}; —Si(R^{f})₂R^{g}, —OR^{b}, —SR^{b}, —S(O)ₘR^{b}, —S(O)ₙN(R^{b})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e};

R^{a} is selected from the group consisting SF₅, C₁-C₆-alkyl, C₁-C₆-fluoroalkyl, C₁-C₆-alkoxy-C₁-C₆-alkyl, C₃-C₈-cycloalkyl, C₃-C₈-fluorocycloalkyl, C₂-C₆-alkenyl, C₂-C₆-fluoroalkenyl, —Si(R^{f})₂R^{g}, —OR^{b}, —SR^{b}, —S(O)ₘR^{b}, —S(O)ₙN(R^{c})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e};

or two geminally bound radicals R^{a} together form a group selected from ═CR^{h}Rⁱ, ═NR^{c1}, ═NOR^{b} and ═NNR^{c1};

or two radicals R^{a}, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;

wherein, in the case of more than one R^{a}, R^{a} can be identical or different;

R^{b} is selected from the group consisting of C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, wherein the six last mentioned radicals may optionally carry 1 or 2 radicals selected from C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-fluoroalkylsulfinyl, C₁-C₆-alkylsulfonyl, C₁-C₆-fluoroalkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;

wherein, in the case of more than one R^{b}, R^{b} can be identical or different;

R^{c}, R^{d} are, independently from one another and independently of each occurrence, selected from the group consisting of cyano, C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, wherein the six last mentioned radicals may optionally carry 1 or 2 radicals selected from C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-alkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;

or R^{c} and R^{d}, together with the nitrogen atom to which they are bound, form a 3-, 4-, 5-, 6- or 7-membered saturated, partly unsaturated or completely unsaturated heterocyclic ring which may contain 1 or 2 further heteroatoms selected from N, O and S as ring members, where the heterocyclic ring may carry 1, 2, 3 or 4 substituents selected from halogen, C₁-C₄-alkyl, C₁-C₄-fluoroalkyl, C₁-C₄-alkoxy and C₁-C₄-fluoroalkoxy;

R^{c1} is hydrogen or has one of the meanings given for R^{c};

R^{d1} is hydrogen or has one of the meanings given for R^{d};

R^{e} is selected from the group consisting of halogen, C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, where the six last mentioned radicals may optionally carry 1 or 2 radicals selected from C₁-C₄-alkoxy; C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-fluoroalkylsulfinyl, C₁-C₆-alkylsulfonyl, C₁-C₆-fluoroalkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;

wherein, in the case of more than one R^{e}, R^{e} can be identical or different;

R^{f}, R^{g} are, independently of each other and independently of each occurrence, selected from the group consisting of C₁-C₄-alkyl, C₃-C₆-cycloalkyl, C₁-C₄-alkoxy-C₁-C₄-alkyl, phenyl and benzyl;

R^{h}, Rⁱ are, independently from one another and independently of each occurrence, selected from the group consisting of hydrogen, halogen, SF₅, C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, where the six last mentioned radicals may optionally carry 1 or 2 radicals selected from C₁-C₄-alkyl and C₁-C₄-fluoroalkyl; C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-alkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy, C₁-C₆-fluoroalkoxy, (C₁-C₆-alkoxy)carbonyl, (C₁-C₆-alkyl)amino and di-(C₁-C₆-alkyl)amino;

m is 1 or 2, wherein, in the case of several occurrences, m may be identical or different;

n is 0, 1 or 2; wherein, in the case of several occurrences, n may be identical or different;

r is 0, 1, 2, 3 or 4;

comprising the steps of
i) reacting a compound of the formula (II)

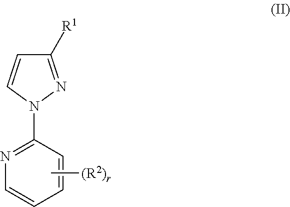

in which the variables R¹, R² and r are each as defined above, with a base selected from combinations of a magnesium-organic compound having a carbon bound magnesium and a secondary amine and magnesium amides of secondary amines in the presence of a lithium halide, where the base is used in an amount sufficient to achieve at least 80% deprotonation of the compound of formula (II); and
ii) subjecting the product obtained in step (i) to a carboxylation by reacting it with carbon dioxide or a carbon dioxide equivalent, to obtain a magnesium salt of the compound of formula (I-A) and optionally aqueous workup to obtain the compound of the formula (I-A) as a free acid.

In case a magnesium salt of the N-substituted 1H-pyrazole-5-carboxylic acid of the formula (I-A) is prepared in the process, the magnesium salt comprises a magnesium cation and the compound of the formula (I-A) in the form of its carboxylate. Preferably, the a magnesium cation is (Mg²⁺)/2, (MgBr⁺) or (MgCl⁺), and in particular is (Mg²⁺)/2

A second aspect of the present invention relates to a process for preparing an N-substituted 1H-pyrazole-5-carbonyl chloride of the formula (I):

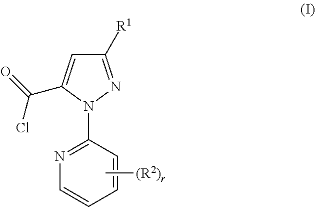

wherein the variables R¹, R² and r are each as defined herein, which comprises
a) preparing an N-substituted 1H-pyrazole-5-carboxylic acid of the formula (I-A) or a magnesium salt thereof by the herein described process and
b) converting the compound of the formula (I-A) or the magnesium salt into the compound of formula (I) by treatment with a chlorinating agent.

The preceding processes of the invention are associated with a series of advantages as they overcome the aforementioned shortcomings of the prior art processes. For instance, the process according to the invention enables the preparation of N-substituted 1H-pyrazole-5-carboxylic acid of the formula (I-A) or its magnesium salt de facto in one process step, since the deprotonated intermediate obtained after reaction step (i) is converted in-situ without prior work-up or purification into the product of formula (I-A) or its magnesium salt. The processes of the invention further enable the preparation of N-substituted 1H-pyrazole-5-carbonylchloride compounds of the formula (I) via the useful intermediates of formula (I-A) or the magnesium salt thereof. The intermediate magnesium salt of the acid I-A can be isolated or can be further converted directly to a compound of formula I or to the free acid of formula (I-A), with or without prior work-up or purification. The intermediate of formula (I-A) can be converted to a compound of formula I with or without prior work-up or purification. If the processes are without work-up or purification steps, the preparation of carbonylchloride compounds of the formula (I) is done de facto in one process step. This prevents losses during work-up or purification and also saves time, resources and/or energy. Also, after completion of the conversion the acid chloride I can be readily isolated and purified by means of a simple protocol including crystallization and solvent evaporation to remove unwanted byproducts. Furthermore, the deprotonation step is carried out with an inexpensive base, which allows for selective, high-yielding and fast conversions at moderate temperatures that can be safely and smoothly carried out on an industrial scale.

Advantages of the processes of the present invention are that the processes allows for moderate temperatures and short reaction times while using safe and inexpensive reagents, which is favorable in view of costs and safety aspects. The yields are generally high and only few by-products, if any, are formed in low amounts, which saves time, resources and energy. Due to these properties, the processes are therefore suitable for an industrial scale, which is a further advantage.

A third aspect of the invention relates to a process for preparing a sulfimine compound of formula (VI)

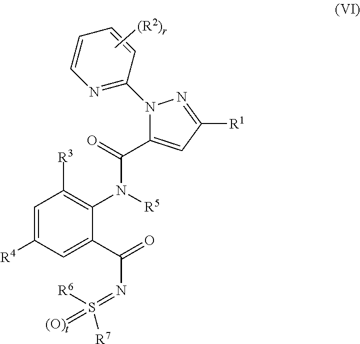

in which

R¹, R² and r are each as defined herein and in the claims;

R³ and R⁴ are independently selected from the group consisting of halogen, cyano, azido, nitro, —SCN, SF₅, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the eight last mentioned radicals may be substituted by one or more radicals R^{a}, —Si(R^{f})₂R^{g}, —OR^{b1}, —OS(O)ₙR^{b1}, SR^{b1}, —S(O)ₘR^{b1}, —S(O)ₘN(R^{b1})R^{d1}, —N(R^{b1})R^{d1}, —N(R^{b1})C(═O)R^{a}, —C(═O)R^{a}, —C(═O)OR^{b1}, —C(═S)R^{a}, —C(═S)OR^{b1}, —C(═NR^{c1})R^{a}, —C(═O)N(R^{c1})R^{d1}, —C(═S)N(R^{c1})R^{d1}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e};

R⁵ is selected from the group consisting of hydrogen; cyano; C₁-C₁₀-alkyl, C₁-C₁₀-haloalkyl, C₃-C₈-cycloalkyl, C₃-C₈-halocycloalkyl, C₂-C₁₀-alkenyl, C₂-C₁₀-haloalkenyl, C₂-C₁₀-alkynyl, C₂-C₁₀-haloalkynyl, wherein the eight last radicals may optionally be substituted by one or more radicals R^{a}; —N(R^{c1})R^{d1}; —Si(R^{f})₂R^{g}; —OR^{b1}; —SR^{b1}; —S(O)ₘR^{b1}; —S(O)ₙN(R^{c1})R^{d1}; —C(═O)R^{a}; —C(═O)OR^{b1}; —C(═O)N(R^{c1})R^{d1}; —C(═S)R^{a}; —C(═S)OR^{b1}; —C(═S)N(R^{c1})R^{d1}; —C(═NR^{c1})R^{a}; phenyl which may be substituted by 1, 2, 3, 4 or 5 radicals R^{e}; and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e};

R⁶ and R⁷ are selected independently of one another from the group consisting of hydrogen, C₁-C₁₀-alkyl, C₁-C₁₀-haloalkyl, C₃-C₈-cycloalkyl, C₃-C₈-halocycloalkyl, C₂-C₁₀-alkenyl, C₂-C₁₀-haloalkenyl, C₂-C₁₀-alkynyl, C₂-C₁₀-haloalkynyl, wherein the eight last radicals may optionally be substituted by one or more radicals R^{a};

or R⁶ and R⁷ together represent a C₂-C₇-alkylene, C₂-C₇-alkenylene or C₂-C₁₀-alkynylene chain forming together with the sulfur atom to which they are attached a 3-, 4-, 5-, 6-, 7-, 8-, 9- or 10-membered saturated, partially unsaturated or completely unsaturated ring, wherein 1 to 4 of the CH₂ groups in the C₂-C₇-alkylene chain or 1 to 4 of any of the CH₂ or CH groups in the C₆-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl; said substituents being identical or different from one another if more than one substituent is present;

R^{a}, R^{c1}, R^{d1}, R^{e}, R^{f}, R^{g}, m and n are each as defined herein and in the claims;

R^{b1} is hydrogen or has one of the meanings given herein and in the claims for R^{b}; and

t is 0 or 1;

which comprises providing a compound of the formula (I) by a process defined herein and in the claims followed by the step of

c) reacting the compound of the formula (I) with a compound of the formula (VII)

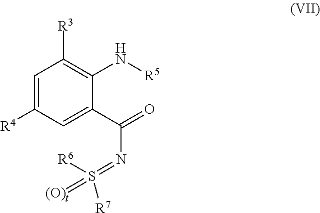

in which the variables R³, R⁴, R⁵, R⁶, R⁷ and t are each as defined above, in the presence of a base, to obtain a compound of the formula (VI).

In the context of the present invention, the terms used generically are each defined as follows:

The prefix Cₓ-C_{y} refers in the particular case to the number of possible carbon atoms.

The term “halogen” denotes in each case fluorine, bromine, chlorine or iodine, in particular fluorine, chlorine or bromine.

The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine.

The term “alkyl” as used herein (and in the alkyl moieties of other groups comprising an alkyl group, e.g. alkoxy, alkylcarbonyl, alkylthio, alkylsulfinyl, alkylsulfonyl and alkoxyalkyl) denotes in each case a straight-chain or branched alkyl group having usually from 1 to 14 carbon atoms, frequently from 1 to 10 carbon atoms, preferably 1 to 6 carbon atoms, more preferably 1 to 4 carbon atoms and in particular from 1 to 3 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, n-octyl, 1-methylheptyl, 2-methylheptyl, 1-ethylhexyl, 2-ethylhexyl, 1,2-dimethylhexyl, 1-propylpentyl, 2-propylpentyl, n-nonyl, 1-methyloctyl, 2-methyloctyl, 2-ethylheptyl, 2-propylhexyl, n-decyl, 1-methylnonyl, 1-ethyloctyl, 2-propylheptyl, 2-butylhexyl, n-undecyl, 1-methyldecyl, 2-methyldecyl, 2-ethylnonyl, 2-propyloctyl, n-dodecyl, 1-methylundecyl, 2-methylundecyl, 2-ethyldecyl, 2-propylnonyl, 2-butyloctyl and the like.

The term “alkylene” (or alkanediyl) as used herein in each case denotes an alkyl radical as defined above, wherein one hydrogen atom at any position of the carbon backbone is replaced by one further binding site, thus forming a bivalent moiety.

The term “haloalkyl” as used herein (and in the haloalkyl moieties of other groups comprising a haloalkyl group, e.g. haloalkoxy and haloalkylthio) denotes in each case a straight-chain or branched alkyl group having usually from 1 to 10 carbon atoms, frequently from 1 to 6 carbon atoms, wherein the hydrogen atoms of this group are partially or totally replaced with halogen atoms. Preferred haloalkyl moieties are selected from C₁-C₄-haloalkyl, more preferably from C₁-C₂-haloalkyl, more preferably from halomethyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “fluoroalkyl”, as used herein (and in the fluoroalkyl units of fluoroalkoxy, fluoroalkylthio, fluoroalkylsulfinyl and fluoroalkylsulfonyl) denotes in each case straight-chain or branched alkyl groups having usually from 1 to 10 carbon atoms, frequently from 1 to 6 carbon atoms and in particular 1 to 4 carbon atoms, wherein the hydrogen atoms of this group are partially or totally replaced with fluorine atoms. Examples thereof are fluoromethyl, difluoromethyl, trifluoromethyl, 1-fluoroethyl, 2-fluoroethyl, 2,2-difluoroethyl, 2,2,2-trifluoroethyl, pentafluoroethyl, 3,3,3-trifluoroprop-1-yl, 1,1,1-trifluoroprop-2-yl, heptafluoroisopropyl, 1-fluorobutyl, 2-fluorobutyl, 3-fluorobutyl, 4-fluorobutyl, 4,4,4-trifluorobutyl, fluoro-tert-butyl and the like.

The term “cycloalkyl” as used herein (and in the cycloalkyl moieties of other groups comprising a cycloalkyl group, e.g. cycloalkoxy and cycloalkylalkyl) denotes in each case a mono- or bicyclic cycloaliphatic radical having usually from 3 to 10 carbon atoms, 3 to 8 carbon atoms or 3 to 6 carbon atoms, such as cyclopropyl, cyclobutyl, cyclopentyl, cyclohexyl, cycloheptyl, cyclooctyl, bicyclo[2.1.1]hexyl, bicyclo[3.1.1]heptyl, bicyclo[2.2.1]heptyl, and bicyclo[2.2.2]octyl.

The term “halocycloalkyl” as used herein (and in the halocycloalkyl moieties of other groups comprising an halocycloalkyl group, e.g. halocycloalkylmethyl) denotes in each case a mono- or bicyclic cycloaliphatic radical having usually from 3 to 10 carbon atoms, 3 to 8 carbon atoms or 3 to 6 carbon atoms, wherein at least one, e.g. 1, 2, 3, 4 or 5 of the hydrogen atoms are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-, 2- and 3-fluorocyclopentyl, 1,2-, 2,2-, 2,3-, 3,3-, 3,4-, 2,5-difluorocyclopentyl, 1-, 2- and 3-chlorocyclopentyl, 1,2-, 2,2-, 2,3-, 3,3-, 3,4-, 2,5-dichlorocyclopentyl and the like.

The term “fluorocylcoalkyl” as used herein, denotes a halocycloalkyl radical, as defined above, wherein the one or more halogen atoms are fluorine atoms.

The term “alkenyl” as used herein denotes in each case a singly unsaturated hydrocarbon radical having usually 2 to 10, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “alkenylene” (or alkenediyl) as used herein in each case denotes an alkenyl radical as defined above, wherein one hydrogen atom at any position of the carbon backbone is replaced by one further binding site, thus forming a bivalent moiety.

The term “haloalkenyl” as used herein, which may also be expressed as “alkenyl which may be substituted by halogen”, and the haloalkenyl moieties in haloalkenyloxy, haloalkenylcarbonyl and the like refers to unsaturated straight-chain or branched hydrocarbon radicals having 2 to 10 (“C₂-C₁₀-haloalkenyl”) or 2 to 6 (“C₂-C₆-haloalkenyl”) carbon atoms and a double bond in any position, where some or all of the hydrogen atoms in these groups are replaced by halogen atoms as mentioned above, in particular fluorine, chlorine and bromine, for example chlorovinyl, chloroallyl and the like.

The term “fluoroalkenyl” as used herein, denotes a haloalkenyl radical, as defined above, wherein the one or more halogen atoms are fluorine atoms.

The term “alkynyl” as used herein denotes unsaturated straight-chain or branched hydrocarbon radicals having usually 2 to 10, frequently 2 to 6, preferably 2 to 4 carbon atoms and one or two triple bonds in any position, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “alkynylene” (or alkynediyl) as used herein in each case denotes an alkynyl radical as defined above, wherein one hydrogen atom at any position of the carbon backbone is replaced by one further binding site, thus forming a bivalent moiety.

The term “haloalkynyl” as used herein, which is also expressed as “alkynyl which may be substituted by halogen”, refers to unsaturated straight-chain or branched hydrocarbon radicals having usually 3 to 10 carbon atoms, frequently 2 to 6, preferably 2 to 4 carbon atoms, and one or two triple bonds in any position (as mentioned above), where some or all of the hydrogen atoms in these groups are replaced by halogen atoms as mentioned above, in particular fluorine, chlorine and bromine.

The term “alkoxy” as used herein denotes in each case a straight-chain or branched alkyl group usually having from 1 to 10 carbon atoms, frequently from 1 to 6 carbon atoms, preferably 1 to 4 carbon atoms, which is bound to the remainder of the molecule via an oxygen atom. Examples of an alkoxy group are methoxy, ethoxy, n-propoxy, iso-propoxy, n-butyloxy, 2-butyloxy, iso-butyloxy, tert-butyloxy, and the like.

The term “haloalkoxy” as used herein denotes in each case a straight-chain or branched alkoxy group, as defined above, having from 1 to 10 carbon atoms, frequently from 1 to 6 carbon atoms, preferably 1 to 4 carbon atoms, preferably 1 to 3 carbon atoms, wherein the hydrogen atoms of this group are partially or totally replaced with halogen atoms, in particular fluorine atoms. Preferred haloalkoxy moieties include C₁-C₄-haloalkoxy, in particular halomethoxy, and also in particular C₁-C₂-fluoroalkoxy, such as fluoromethoxy, difluoromethoxy, trifluoromethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2-dichloro-2-fluorethoxy, 2,2,2-trichloroethoxy, pentafluoroethoxy and the like.

The term “alkoxy-alkyl” as used herein denotes in each case alkyl usually comprising 1 to 6 carbon atoms, preferably 1 to 4 carbon atoms, wherein 1 carbon atom carries an alkoxy radical usually comprising 1 to 10, frequently 1 to 6, in particular 1 to 4, carbon atoms as defined above. Examples are CH₂OCH₃, CH₂—OC₂H₅, n-propoxymethyl, CH₂—OCH(CH₃)₂, n-butoxymethyl, (1-methylpropoxy)-methyl, (2-methylpropoxy)methyl, CH₂—OC(CH₃)₃, 2-(methoxy)ethyl, 2-(ethoxy)ethyl, 2-(n-propoxy)-ethyl, 2-(1-methylethoxy)-ethyl, 2-(n-butoxy)ethyl, 2-(1-methylpropoxy)-ethyl, 2-(2-methylpropoxy)-ethyl, 2-(1,1-dimethylethoxy)-ethyl, 2-(methoxy)-propyl, 2-(ethoxy)-propyl, 2-(n-propoxy)-propyl, 2-(1-methylethoxy)-propyl, 2-(n-butoxy)-propyl, 2-(1-methylpropoxy)-propyl, 2-(2-methylpropoxy)-propyl, 2-(1,1-dimethylethoxy)-propyl, 3-(methoxy)-propyl, 3-(ethoxy)-propyl, 3-(n-propoxy)-propyl, 3-(1-methylethoxy)-propyl, 3-(n-butoxy)-propyl, 3-(1-methylpropoxy)-propyl, 3-(2-methylpropoxy)-propyl, 3-(1,1-dimethylethoxy)-propyl, 2-(methoxy)-butyl, 2-(ethoxy)-butyl, 2-(n-propoxy)-butyl, 2-(1-methylethoxy)-butyl, 2-(n-butoxy)-butyl, 2-(1-methylpropoxy)-butyl, 2-(2-methyl-propoxy)-butyl, 2-(1,1-dimethylethoxy)-butyl, 3-(methoxy)-butyl, 3-(ethoxy)-butyl, 3-(n-propoxy)-butyl, 3-(1-methylethoxy)-butyl, 3-(n-butoxy)-butyl, 3-(1-methylpropoxy)-butyl, 3-(2-methylpropoxy)-butyl, 3-(1,1-dimethylethoxy)-butyl, 4-(methoxy)-butyl, 4-(ethoxy)-butyl, 4-(n-propoxy)-butyl, 4-(1-methylethoxy)-butyl, 4-(n-butoxy)-butyl, 4-(1-methylpropoxy)-butyl, 4-(2-methylpropoxy)-butyl, 4-(1,1-dimethylethoxy)-butyl and the like.

The term “fluoroalkoxy-alkyl” as used herein denotes in each case alkyl as defined above, usually comprising 1 to 6 carbon atoms, preferably 1 to 4 carbon atoms, wherein 1 carbon atom carries an fluoroalkoxy radical as defined above, usually comprising 1 to 10, frequently 1 to 6, in particular 1 to 4, carbon atoms as defined above. Examples are fluoromethoxymethyl, difluoromethoxymethyl, trifluoromethoxymethyl, 1-fluoroethoxymethyl, 2-fluoroethoxymethyl, 1,1-difluoroethoxymethyl, 1,2-difluoroethoxymethyl, 2,2-difluoroethoxymethyl, 1,1,2-trifluoroethoxymethyl, 1,2,2-trifluoroethoxymethyl, 2,2,2-trifluoroethoxymethyl, pentafluoroethoxymethyl, 1-fluoroethoxy-1-ethyl, 2-fluoroethoxy-1-ethyl, 1,1-difluoroethoxy-1-ethyl, 1,2-difluoroethoxy-1-ethyl, 2,2-difluoroethoxy-1-ethyl, 1,1,2-trifluoroethoxy-1-ethyl, 1,2,2-trifluoroethoxy-1-ethyl, 2,2,2-trifluoroethoxy-1-ethyl, pentafluoroethoxy-1-ethyl, 1-fluoroethoxy-2-ethyl, 2-fluoroethoxy-2-ethyl, 1,1-difluoroethoxy-2-ethyl, 1,2-difluoroethoxy-2-ethyl, 2,2-difluoroethoxy-2-ethyl, 1,1,2-trifluoroethoxy-2-ethyl, 1,2,2-trifluoroethoxy-2-ethyl, 2,2,2-trifluoroethoxy-2-ethyl, pentafluoroethoxy-2-ethyl, and the like.

The term “alkylthio” (also alkylsulfanyl or alkyl-S—)” as used herein denotes in each case a straight-chain or branched saturated alkyl group as defined above, usually comprising 1 to 10 carbon atoms, frequently comprising 1 to 6 carbon atoms, preferably 1 to 4 carbon atoms, which is attached via a sulfur atom at any position in the alkyl group. Examples are methylthio, ethylthio, n-propylthio, iso-propylthio, n-butylthio, 2-butylthio, iso-butylthio, tert-butylthio, and the like.

The term “haloalkylthio” as used herein refers to an alkylthio group as defined above wherein the hydrogen atoms are partially or fully substituted by fluorine, chlorine, bromine and/or iodine. Examples are fluoromethylthio, difluoromethylthio, trifluoromethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoro-ethylthio, 2,2-dichloro-2-fluorethylthio, 2,2,2-trichloroethylthio, pentafluoroethylthio and the like

The terms “alkylsulfinyl” and “S(O)ₙ-alkyl” (wherein n is 1) are equivalent and, as used herein, denote an alkyl group, as defined above, attached via a sulfinyl [S(O)] group. For example, the term “C₁-C₆-alkylsulfinyl” refers to a C₁-C₆-alkyl group, as defined above, attached via a sulfinyl [S(O)] group. Examples are methylsulfinyl, ethylsulfinyl, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutylsulfinyl), 1,1-dimethylethylsulfinyl (tert-butylsulfinyl), pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl.

The terms “alkylsulfonyl” and “S(O)ₙ-alkyl” (wherein n is 2) are equivalent and, as used herein, denote an alkyl group, as defined above, attached via a sulfonyl [S(O)₂] group. For example, the term “C₁-C₆-alkylsulfonyl” refers to a C₁-C₆-alkyl group, as defined above, attached via a sulfonyl [S(O)₂] group. Examples are methylsulfonyl, ethylsulfonyl, n-propylsulfonyl, 1-methylethylsulfonyl (isopropylsulfonyl), butylsulfonyl, 1-methylpropylsulfonyl (sec-butylsulfonyl), 2-methylpropylsulfonyl (isobutylsulfonyl), 1,1-dimethylethylsulfonyl (tert-butylsulfonyl), pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-d imethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropyl-sulfonyl and 1-ethyl-2-methylpropylsulfonyl.

The term “alkylamino” as used herein denotes in each case a group —NHR, wherein R is a straight-chain or branched alkyl group usually having from 1 to 6 carbon atoms, preferably 1 to 4 carbon atoms. Examples of an alkylamino group are methylamino, ethylamino, n-propylamino, isopropylamino, n-butylamino, 2-butylamino, iso-butylamino, tert-butylamino, and the like.

The term “dialkylamino” as used herein denotes in each case a group-NRR′, wherein R and R′, independently of each other, are a straight-chain or branched alkyl group each usually having from 1 to 6 carbon atoms, preferably 1 to 4 carbon atoms. Examples of a dialkylamino group are dimethylamino, diethylamino, dipropylamino, dibutylamino, methyl-ethyl-amino, methyl-propyl-amino, methyl-isopropylamino, methyl-butyl-amino, methyl-isobutyl-amino, ethyl-propyl-amino, ethyl-isopropylamino, ethyl-butyl-amino, ethyl-isobutyl-amino, and the like.

The suffix “-carbonyl” in a group denotes in each case that the group is bound to the remainder of the molecule via a carbonyl C═O group. This is the case e.g. in alkylcarbonyl, haloalkylcarbonyl, alkoxycarbonyl and haloalkoxycarbonyl.

The term “aryl” as used herein refers to a mono-, bi- or tricyclic aromatic hydrocarbon radical having 6 to 14 carbon atoms. Examples thereof comprise phenyl, naphthyl, fluorenyl, azulenyl, anthracenyl and phenanthrenyl. Aryl is preferably phenyl or naphthyl and especially phenyl.

The term “3-, 4-, 5-, 6-, 7- or 8-membered saturated carbocyclic ring” as used herein refers to carbocyclic rings, which are monocyclic and fully saturated. Examples of such rings include cyclopropane, cyclobutane, cyclopentane, cyclohexane, cycloheptane, cyclooctane and the like.

The terms “3-, 4-, 5-, 6-, 7- or 8-membered partially unsaturated carbocyclic ring” and “5- or 6-membered partially unsaturated carbocyclic ring” refer to carbocyclic rings, which are monocyclic and have one or more degrees of unsaturation. Examples of such rings include include cyclopropene, cyclobutene, cyclopentene, cyclohexene, cycloheptene, cyclooctene and the like.

The term “3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members” [wherein “completely/fully unsaturated” includes also “aromatic”] as used herein denotes monocyclic radicals, the monocyclic radicals being saturated, partially unsaturated or fully unsaturated (including aromatic). The heterocyclic ring may be attached to the remainder of the molecule via a carbon ring member or via a nitrogen ring member.

Examples of a 3-, 4-, 5-, 6- or 7-membered saturated heterocyclic ring include: oxiranyl, aziridinyl, azetidinyl, tetrahydrofuran-2-yl, tetrahydrofuran-3-yl, tetrahydrothien-2-yl, tetrahydrothien-3-yl, pyrrolidin-2-yl, pyrrolidin-3-yl, pyrazolidin-3-yl, pyrazolidin-4-yl, pyrazolidin-5-yl, imidazolidin-2-yl, imidazolidin-4-yl, oxazolidin-2-yl, oxazolidin-4-yl, oxazolidin-5-yl, isoxazolidin-3-yl, isoxazolidin-4-yl, isoxazolidin-5-yl, thiazolidin-2-yl, thiazolidin-4-yl, thiazolidin-5-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2-tetrahydropyranyl, 4-tetrahydropyranyl, 1,3-dioxan-5-yl, 1,4-dioxan-2-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-2-yl, 1,3,5-hexahydrotriazin-2-yl and 1,2,4-hexahydrotriazin-3-yl, morpholin-2-yl, morpholin-3-yl, thiomorpholin-2-yl, thiomorpholin-3-yl, 1-oxothiomorpholin-2-yl, 1-oxothiomorpholin-3-yl, 1,1-dioxothiomorpholin-2-yl, 1,1-dioxothiomorpholin-3-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.
Examples of a 3-, 4-, 5-, 6- or 7-membered partially unsaturated heterocyclic ring include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.
A 3-, 4-, 5-, 6- or 7-membered completely unsaturated (including aromatic) heterocyclic ring is e.g. a 5- or 6-membered fully unsaturated (including aromatic) heterocyclic ring. Examples are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 4-isoxazolyl, 2-thiazolyl, 4-thiazolyl, 5-thiazolyl, 4-isothiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

The term “a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members” as used herein denotes a saturated or unsaturated 3- to 8-membered ring system which optionally contains 1 to 3 heteroatoms selected from N, O, S, NO, SO and SO₂, as defined above, with the exception of the completely unsaturated ring systems.

The remarks made below concerning preferred embodiments of the variables of the compounds of the formulae (I), (I-A), (II), (VI) and (VII) are valid on their own as well as preferably in combination with each other concerning the methods according to the invention.

In the compounds of the formulae (I), (I-A), (II) and (VI), R¹ is preferably an electron-withdrawing group and is preferably selected from halogen, C₁-C₄-alkyl, C₁-C₄-fluoroalkyl, CBrF₂, C₅-C₆-cycloalkyl, C₅-C₆-fluorocycloalkyl, C₂-C₄-alkenyl, C₂-C₄-fluoroalkenyl, wherein the six last mentioned radicals may be substituted by 1, 2 or 3 radicals R^{a}; —OR^{b}, —SR^{b}, —N(R^{c1})R^{d1}, phenyl which may be substituted by 1, 2 or 3 radicals R^{e}, and a 5- or 6-membered saturated, partially unsaturated or aromatic heterocyclic ring containing 1 or 2 heteroatoms or heteroatom groups selected from N, O and S as ring members, where the heterocyclic ring may be substituted by 1, 2 or 3 radicals R^{e}. In a specific embodiment, R¹ is as defined herein and in the claims, with the proviso, that it is not CBrF₂.

More preferably R¹ is selected from fluorine, chlorine, C₁-C₄-fluoroalkyl, C₁-C₄-alkoxy and C₁-C₄-fluoroalkoxy-C₁-C₄-alkyl, particularly selected from fluorine, chlorine, CF₃, CHF₂ and methoxy, and specifically from CF₃ and CHF₂.

In the compounds of the formulae (I), (I-A), (II) and (VI), each R² preferably is independently selected from halogen, C₁-C₄-alkyl, C₁-C₄-fluoroalkyl, C₅-C₆-cycloalkyl, C₅-C₆-fluorocycloalkyl, C₂-C₄-alkenyl, C₂-C₄-fluoroalkenyl, wherein the six last mentioned radicals may be substituted by one or more radicals R^{a}; —OR^{b}, —SR^{b}, —N(R^{c1})R^{d1}, phenyl which may be substituted by 1, 2 or 3 radicals R^{e}, and a 5- or 6-membered saturated, partially unsaturated or completely unsaturated heterocyclic ring containing 1 or 2 heteroatoms or heteroatom groups selected from N, O and S, as ring members, where the heterocyclic ring may be substituted by 1, 2 or 3 radicals R^{e}.

More preferably each R² is independently selected from halogen and halomethyl, in particular from halogen and CF₃ and specifically R² is chlorine.

In the compounds of the formulae (I), (I-A), (II) and (VI), r is preferably 1, 2 or 3 and more preferably 1. When r is 1, R² is preferably located in position 3 of the pyridyl moiety of the compound of the formulae (I), (I-A), (II) or (VI), i.e. is bound to the ring carbon atom of the pyridyl moiety that is ortho to the pyrazole bond.

N-substituted 1H-pyrazole-5-carboxylic acid of the formula (I-A) are known, e.g. from WO 02/070483 or WO03/015519.

In the compounds of the formulae (VI) and (VII), R³ and R⁴ are preferably, independently of each other, selected from halogen, cyano, nitro, C₁-C₄-alkyl, haloalkyl, C₅-C₆-cycloalkyl, C₅-C₈-halocycloalkyl, C₂-C₄-alkenyl, C₂-C₄-haloalkenyl, wherein the six last mentioned radicals may be substituted by one or more radicals R^{a}; —OR^{b1}, —OS(O)ₙR^{b1}, SR^{b1}, —N(R^{b1})R^{d1}, —C(═O)R^{a}, phenyl which may be substituted by 1, 2 or 3 radicals R^{e}, and a 5- or 6-membered saturated, partially unsaturated or completely unsaturated heterocyclic ring containing 1 or 2 heteroatoms selected from N, O and S, as ring members, where the heterocyclic ring may be substituted by 1, 2 or 3 radicals R^{e}.

More preferably R³ and R⁴ are independently selected from halogen, cyano, C₁-C₄-alkyl and C₁-C₄-haloalkyl. Particularly preferred R³ is selected from halogen, methyl and halomethyl, in particular from chlorine, bromine, methyl, CF₃ and CHF₂, specifically from chlorine, bromine, methyl, and R⁴ is selected from halogen, cyano, methyl and halomethyl, in particular from specifically from chlorine, bromine, cyano, CF₃ and CHF₂, specifically from chlorine, bromine and cyano.

In the compounds of the formulae (VI) and (VII), R⁵ is preferably selected from hydrogen, C₁-C₆-alkyl, C₁-C₆-haloalkyl, C₅-C₆-cycloalkyl, C₅-C₆-halocycloalkyl, wherein the four last radicals may optionally be substituted by one or more radicals R^{a}; —C(═O)R^{a}; phenyl which may be substituted by 1, 2 or 3 radicals R^{e}; and a 5- or 6-membered saturated, partially unsaturated or completely unsaturated heterocyclic ring containing 1 or 2 heteroatoms selected from N, O and S, as ring members, where the heterocyclic ring may be substituted by 1, 2 or 3 radicals R^{e}.

More preferably each R⁵ is selected from hydrogen, C₁-C₄-alkyl, C₁-C₄-haloalkyl and —C(═O)—C₁-C₄-alkyl, in particular from hydrogen, C₁-C₃-alkyl and halomethyl, and specifically R⁵ is hydrogen.

In the compounds of the formulae (VI) and (VII), t is preferably 0. In the compounds of the formulae (VI) and (VII), wherein t is 0, R⁶ and R⁷ are preferably, independently of each other, selected from hydrogen, C₁-C₆-alkyl, C₁-C₆-haloalkyl, C₃-C₆-cycloalkyl, C₃-C₆-halocycloalkyl, C₂-C₄-alkenyl, C₂-C₄-haloalkenyl, wherein the six last radicals may optionally be substituted by one or more radicals R^{a};

or R⁶ and R⁷ together represent a C₃-C₆-alkylene or C₃-C₆-alkenylene chain forming together with the sulfur atom to which they are attached a 4-, 5-. 6- or 7-membered saturated or partially unsaturated ring, wherein one of the CH₂ groups in the C₄-C₅-alkylene chain or one of the CH₂ or CH groups in the C₄-C₅-alkenylene chain may be replaced by a group independently selected from O, S and N and NH, and wherein the carbon and/or nitrogen atoms in the C₃-C₅-alkylene or C₃-C₆-alkenylene chain may be substituted with 1 or 2 substituents independently selected from halogen, cyano, C₁-C₄-alkoxy, C₁-C₄-haloalkoxy.

More preferably R⁶ and R⁷ are independently selected from C₁-C₆-alkyl, C₁-C₆-haloalkyl, or R⁶ and R⁷ together represent a C₃-C₆-alkylene chain forming together with the sulfur atom to which they are attached a 4-, 5-. 6- or 7-membered ring. Particularly preferred R⁶ and R⁷ are each C₁-C₆-alkyl, or together represent a C₃-C₆-alkylene chain forming together with the sulfur atom to which they are attached a 4-, 5-. 6- or 7-membered ring. More preferably R⁶ and R⁷ are independently selected from C₁-C₄-alkyl, or R⁶ and R⁷ together represent a C₄-C₅-alkylene chain forming together with the sulfur atom to which they are attached a 5- or 6-membered ring. Even more preferably R⁶ and R⁷ are independently selected from C₁-C₄-alkyl. Particularly preferred, when t is 0, Wand R⁷ are selected independently of one another from C₁-C₆-alkyl, or R⁶ and together represent a C₃-C₆-alkylene chain forming together with the sulfur atom to which they are attached a 4-, 5-, 6- or 7-membered saturated ring. Specifically R⁶ and R⁷ are each methyl, isopropyl or ethyl.

In the compounds of the formulae (VI) and (VII), wherein t is 1, the preferred meanings of R⁶ and R⁷ are the preferred meanings as described above in the compounds of the formulae (VI) and (VII), wherein t is 0.

In this context, the variables R^{a}, R^{b}, R^{c}, R^{d}, R^{b1}, R^{c1}, R^{d1}, R^{e}, R^{f}, R^{g}, R^{h}, m and n, independently of each other, preferably have one of the following meanings:

R^{a} is selected from C₁-C₄-alkyl, C₁-C₄-fluoroalkyl, C₃-C₆-cycloalkyl, C₃-C₆-fluorocycloalkyl, C₂-C₄-alkenyl, C₂-C₄-fluoroalkenyl, C₁-C₄-alkoxy, amino, di-(C₁-C₄-alkyl)-amino, phenyl and a 5- or 6-membered saturated, partially unsaturated or completely unsaturated heterocyclic ring containing 1 or 2 heteroatoms selected from N, O and S, as ring members, where phenyl and the heterocyclic ring may be substituted by 1, 2 or 3 radicals selected from C₁-C₄-alkyl, C₁-C₄-fluoroalkyl, C₅-C₆-cycloalkyl and C₅-C₆-fluorocycloalkyl.

More preferably R^{a} is selected from C₁-C₄-alkyl and C₁-C₄-fluoroalkyl, C₁-C₄-alkoxy, di-(C₁-C₄-alkyl)-amino, phenyl and a 5- or 6-membered saturated, partially unsaturated or completely unsaturated heterocyclic ring containing 1 or 2 heteroatoms selected from N, O and S, as ring members, and in particular selected from C₁-C₃-alkyl and C₁-C₂-fluoroalkyl and C₁-C₂-alkoxy.

R^{b} is selected from C₁-C₄-alkyl, C₁-C₄-fluoroalkyl, C₅-C₆-cycloalkyl, C₅-C₆-fluorocycloalkyl, C₁-C₄-fluoroalkoxy-C₁-C₄-alkyl, phenyl-C₁-C₄-alkyl, phenoxy-C₁-C₄-alkyl and pyridyl-C₁-C₄-alkyl, wherein phenyl and pyridyl in the three last mentioned radicals may optionally carry 1 or 2 radicals selected from halogen, substituents C₁-C₂-fluoroalkyl, C₁-C₄-alkoxy and C₁-C₂-fluoroalkoxy.

More preferably R^{b} is selected from C₁-C₄-alkyl, C₁-C₄-fluoroalkyl and benzyl, and in particular selected from C₁-C₃-alkyl, C₁-C₂-fluoroalkyl and benzyl.

R^{c}, R^{d} are, independently from one another and independently of each occurrence, selected from C₁-C₄-alkyl, C₁-C₄-fluoroalkyl, C₅-C₆-cycloalkyl, C₅-C₆-fluorocycloalkyl, wherein the four last mentioned radicals may optionally carry 1 or 2 radicals selected from C₁-C₄-alkoxy, C₁-C₄-fluoroalkoxy, C₁-C₄-alkylthio, C₁-C₄-fluoroalkylthio, phenyl, benzyl, pyridyl and phenoxy, wherein the four last mentioned radicals may carry 1 or 2 substituents selected from halogen, C₁-C₄-alkyl, C₁-C₂-fluoroalkyl, C₁-C₄-alkoxy and C₁-C₂-fluoroalkoxy; or R^{c} and R^{d}, together with the nitrogen atom to which they are bound, form a 5- or 6-membered saturated, partly unsaturated or completely unsaturated heterocyclic ring which may contain 1 further heteroatom selected from N, O and S as ring members, where the heterocyclic ring may carry 1 or 2 substituents selected from halogen, C₁-C₄-alkyl and C₁-C₄-fluoroalkyl.

More preferably R^{d}, R^{d} are, independently from one another and independently of each occurrence, selected from C₁-C₄-alkyl, C₁-C₄-fluoroalkyl and benzyl, or R^{c} and R^{d}, together with the nitrogen atom to which they are bound, form a 5- or 6-membered saturated or partly unsaturated heterocyclic ring. In particular, R^{d}, R^{d} are, independently from one another and independently of each occurrence, C₁-C₃-alkyl, C₁-C₂-fluoroalkyl, benzyl, or together with the nitrogen atom to which they are bound form a pyrrolidine or a piperidine ring.

R^{b1} is hydrogen or has one of the preferred meanings given for R^{c}.

R^{c1} is hydrogen or has one of the preferred meanings given for R^{c}.

R^{d1} is hydrogen or has one of the preferred meanings given for R^{d}.

R^{e} is selected from halogen, C₁-C₄-alkyl, C₁-C₄-fluoroalkyl, C₂-C₄-alkenyl, C₂-C₄-fluoroalkenyl, where the four last mentioned radicals may optionally carry 1 or 2 radicals selected from C₁-C₂-alkoxy; C₁-C₄-alkoxy, C₁-C₄-fluoroalkoxy, phenyl, benzyl, pyridyl and phenoxy, wherein the four last mentioned radicals may carry 1 or 2 substituents selected from halogen, C₁-C₂-alkyl and C₁-C₂-fluoroalkyl.

More preferably R^{e} is selected from C₁-C₄-alkyl, C₁-C₄-fluoroalkyl, C₁-C₄-alkoxy and C₁-C₄-fluoroalkoxy, and in particular from C₁-C₃-alkyl, C₁-C₂-fluoroalkyl, C₁-C₂-alkoxy, C₁-C₂-fluoroalkoxy.

R^{f}, R^{g} are, independently of each other and independently of each occurrence, selected from C₁-C₄-alkyl, C₅-C₆-cycloalkyl, C₁-C₂-alkoxy-C₁-C₂-alkyl, phenyl and benzyl.

More preferably R^{f}, R^{g} are, independently of each other and independently of each occurrence, selected from C₁-C₄-alkyl, C₅-C₆-cycloalkyl, benzyl and phenyl, and in particular from C₁-C₃-alkyl, benzyl and phenyl.

R^{h}, Rⁱ are, independently from one another and independently of each occurrence, selected from hydrogen, halogen, C₁-C₄-alkyl, C₁-C₄-fluoroalkyl, C₅-C₆-cycloalkyl, C₅-C₆-fluorocycloalkyl, where the four last mentioned radicals may optionally carry 1 or 2 radicals selected from C₁-C₃-alkyl and C₁-C₃-fluoroalkyl; C₁-C₄-alkoxy, C₁-C₄-fluoroalkoxy, phenyl, pyridyl and phenoxy.

More preferably R^{h}, Rⁱ are, independently of each other and independently of each occurrence, selected from hydrogen, C₁-C₃-alkyl and C₁-C₂-fluoroalkyl.

m is 1 or 2, wherein, in the case of several occurrences, m may be identical or different. More preferably m is 2.

n is 1 or 2, wherein, in the case of several occurrences, n may be identical or different. More preferably n is 2.

In the processes according to the first, second and third aspect of the invention particular preference is given to compounds of the formulae (I), (I-A), (II), (VI) and (VII), where R¹ is selected from the group consisting of fluorine, chlorine, C₁-C₄-fluoroalkyl, C₁-C₄-alkoxy and C₁-C₄-fluoroalkoxy-C₁-C₄-alkyl, and in particular selected from fluorine, chlorine, CF₃, CHF₂ and methoxy, r is 1, and R² is located in position 3 of the pyridyl moiety and is selected from halogen and CF₃, and in particular is chlorine.

In the process according to the third aspect of the invention particular preference is given to compounds of the formulae (VI) and (VII), where:
R³ is selected from the group consisting of methyl and halogen, and in particular form the group consisting of methyl, chlorine and bromine;
R⁴ is selected from the group consisting of cyano, methyl and halogen, and in particular form the group consisting of cyano, chlorine and bromine;
R⁵ is hydrogen;
R⁶ and R⁷ are independently of each other selected from C₁-C₄-alkyl, and in particular form the group consisting of methyl, ethyl and isopropyl;
r 1; and
t 0.

In the process according to the first and second aspects of the invention specific preference is given to the compounds of formulae (I), (I-A) and (II) in which r is 1, R² is chlorine and is located in position 3 of the pyridyl moiety, and R¹ selected from fluorine, chlorine, CF₃, CHF₂ and methoxy.

In the process according to the third aspect of the invention specific preference is given to the compounds of formula (VII) in which t is 0, R⁵ is hydrogen, and the combination of R³, R⁴, R⁶ and R⁷ for a compound corresponds in each case to the meanings given for these variables in one of the rows A-1 to A-45 of Table A (compounds VII-1 to VII-45).

In the process according to the third aspect of the invention specific preference is given to the compounds of formula (VI) in which r is 1, t is 0, R⁵ is hydrogen, R² is chlorine and is located in position 3 of the pyridyl moiety, and the combination of R¹, R³, R⁴, R⁶ and R⁷ for a compound corresponds in each case to the meanings given for these variables in one of the rows A-1 to A-225 of Table A (compounds VI-1 to VI-225).

TABLE A

| | R¹ | R³ | R⁴ | R⁶ | R⁷ |
|---|---|---|---|---|---|
| A-1 | fluorine | methyl | chlorine | methyl | methyl |
| A-2 | fluorine | methyl | chlorine | ethyl | methyl |
| A-3 | fluorine | methyl | chlorine | isopropryl | methyl |
| A-4 | fluorine | methyl | chlorine | methyl | ethyl |
| A-5 | fluorine | methyl | chlorine | ethyl | ethyl |
| A-6 | fluorine | methyl | chlorine | isopropryl | ethyl |
| A-7 | fluorine | methyl | chlorine | methyl | isopropryl |
| A-8 | fluorine | methyl | chlorine | ethyl | isopropryl |
| A-9 | fluorine | methyl | chlorine | isopropryl | isopropryl |
| A-10 | fluorine | chlorine | chlorine | methyl | methyl |
| A-11 | fluorine | chlorine | chlorine | ethyl | methyl |
| A-12 | fluorine | chlorine | chlorine | isopropryl | methyl |
| A-13 | fluorine | chlorine | chlorine | methyl | ethyl |
| A-14 | fluorine | chlorine | chlorine | ethyl | ethyl |
| A-15 | fluorine | chlorine | chlorine | isopropryl | ethyl |
| A-16 | fluorine | chlorine | chlorine | methyl | isopropryl |
| A-17 | fluorine | chlorine | chlorine | ethyl | isopropryl |
| A-18 | fluorine | chlorine | chlorine | isopropryl | isopropryl |
| A-19 | fluorine | methyl | cyano | methyl | methyl |
| A-20 | fluorine | methyl | cyano | ethyl | methyl |
| A-21 | fluorine | methyl | cyano | isopropryl | methyl |
| A-22 | fluorine | methyl | cyano | methyl | ethyl |
| A-23 | fluorine | methyl | cyano | ethyl | ethyl |
| A-24 | fluorine | methyl | cyano | isopropryl | ethyl |
| A-25 | fluorine | methyl | cyano | methyl | isopropryl |
| A-26 | fluorine | methyl | cyano | ethyl | isopropryl |
| A-27 | fluorine | methyl | cyano | isopropryl | isopropryl |
| A-28 | fluorine | chlorine | bromine | methyl | methyl |
| A-29 | fluorine | chlorine | bromine | ethyl | methyl |
| A-30 | fluorine | chlorine | bromine | isopropryl | methyl |
| A-31 | fluorine | chlorine | bromine | methyl | ethyl |
| A-32 | fluorine | chlorine | bromine | ethyl | ethyl |
| A-33 | fluorine | chlorine | bromine | isopropryl | ethyl |
| A-34 | fluorine | chlorine | bromine | methyl | isopropryl |
| A-35 | fluorine | chlorine | bromine | ethyl | isopropryl |
| A-36 | fluorine | chlorine | bromine | isopropryl | isopropryl |
| A-37 | fluorine | bromine | bromine | methyl | methyl |
| A-38 | fluorine | bromine | bromine | ethyl | methyl |
| A-39 | fluorine | bromine | bromine | isopropryl | methyl |
| A-40 | fluorine | bromine | bromine | methyl | ethyl |
| A-41 | fluorine | bromine | bromine | ethyl | ethyl |
| A-42 | fluorine | bromine | bromine | isopropryl | ethyl |
| A-43 | fluorine | bromine | bromine | methyl | isopropryl |
| A-44 | fluorine | bromine | bromine | ethyl | isopropryl |
| A-45 | fluorine | bromine | bromine | isopropryl | isopropryl |
| A-46 | chlorine | methyl | chlorine | methyl | methyl |
| A-47 | chlorine | methyl | chlorine | ethyl | methyl |
| A-48 | chlorine | methyl | chlorine | isopropryl | methyl |
| A-49 | chlorine | methyl | chlorine | methyl | ethyl |
| A-50 | chlorine | methyl | chlorine | ethyl | ethyl |
| A-51 | chlorine | methyl | chlorine | isopropryl | ethyl |
| A-52 | chlorine | methyl | chlorine | methyl | isopropryl |
| A-53 | chlorine | methyl | chlorine | ethyl | isopropryl |
| A-54 | chlorine | methyl | chlorine | isopropryl | isopropryl |
| A-55 | chlorine | chlorine | chlorine | methyl | methyl |
| A-56 | chlorine | chlorine | chlorine | ethyl | methyl |
| A-57 | chlorine | chlorine | chlorine | isopropryl | methyl |
| A-58 | chlorine | chlorine | chlorine | methyl | ethyl |
| A-59 | chlorine | chlorine | chlorine | ethyl | ethyl |
| A-60 | chlorine | chlorine | chlorine | isopropryl | ethyl |
| A-61 | chlorine | chlorine | chlorine | methyl | isopropryl |
| A-62 | chlorine | chlorine | chlorine | ethyl | isopropryl |
| A-63 | chlorine | chlorine | chlorine | isopropryl | isopropryl |
| A-64 | chlorine | methyl | cyano | methyl | methyl |
| A-65 | chlorine | methyl | cyano | ethyl | methyl |
| A-66 | chlorine | methyl | cyano | isopropryl | methyl |
| A-67 | chlorine | methyl | cyano | methyl | ethyl |
| A-68 | chlorine | methyl | cyano | ethyl | ethyl |
| A-69 | chlorine | methyl | cyano | isopropryl | ethyl |
| A-70 | chlorine | methyl | cyano | methyl | isopropryl |
| A-71 | chlorine | methyl | cyano | ethyl | isopropryl |
| A-72 | chlorine | methyl | cyano | isopropryl | isopropryl |
| A-73 | chlorine | chlorine | bromine | methyl | methyl |
| A-74 | chlorine | chlorine | bromine | ethyl | methyl |
| A-75 | chlorine | chlorine | bromine | isopropryl | methyl |
| A-76 | chlorine | chlorine | bromine | methyl | ethyl |
| A-77 | chlorine | chlorine | bromine | ethyl | ethyl |
| A-78 | chlorine | chlorine | bromine | isopropryl | ethyl |
| A-79 | chlorine | chlorine | bromine | methyl | isopropryl |
| A-80 | chlorine | chlorine | bromine | ethyl | isopropryl |
| A-81 | chlorine | chlorine | bromine | isopropryl | isopropryl |
| A-82 | chlorine | bromine | bromine | methyl | methyl |
| A-83 | chlorine | bromine | bromine | ethyl | methyl |
| A-84 | chlorine | bromine | bromine | isopropryl | methyl |
| A-85 | chlorine | bromine | bromine | methyl | ethyl |
| A-86 | chlorine | bromine | bromine | ethyl | ethyl |
| A-87 | chlorine | bromine | bromine | isopropryl | ethyl |
| A-88 | chlorine | bromine | bromine | methyl | isopropryl |
| A-89 | chlorine | bromine | bromine | ethyl | isopropryl |
| A-90 | chlorine | bromine | bromine | isopropryl | isopropryl |
| A-91 | CF₃ | methyl | chlorine | methyl | methyl |
| A-92 | CF₃ | methyl | chlorine | ethyl | methyl |
| A-93 | CF₃ | methyl | chlorine | isopropryl | methyl |
| A-94 | CF₃ | methyl | chlorine | methyl | ethyl |
| A-95 | CF₃ | methyl | chlorine | ethyl | ethyl |
| A-96 | CF₃ | methyl | chlorine | isopropryl | ethyl |
| A-97 | CF₃ | methyl | chlorine | methyl | isopropryl |
| A-98 | CF₃ | methyl | chlorine | ethyl | isopropryl |
| A-99 | CF₃ | methyl | chlorine | isopropryl | isopropryl |
| A-100 | CF₃ | chlorine | chlorine | methyl | methyl |
| A-101 | CF₃ | chlorine | chlorine | ethyl | methyl |
| A-102 | CF₃ | chlorine | chlorine | isopropryl | methyl |
| A-103 | CF₃ | chlorine | chlorine | methyl | ethyl |
| A-104 | CF₃ | chlorine | chlorine | ethyl | ethyl |
| A-105 | CF₃ | chlorine | chlorine | isopropryl | ethyl |
| A-106 | CF₃ | chlorine | chlorine | methyl | isopropryl |
| A-107 | CF₃ | chlorine | chlorine | ethyl | isopropryl |
| A-108 | CF₃ | chlorine | chlorine | isopropryl | isopropryl |
| A-109 | CF₃ | methyl | cyano | methyl | methyl |
| A-110 | CF₃ | methyl | cyano | ethyl | methyl |
| A-111 | CF₃ | methyl | cyano | isopropryl | methyl |
| A-112 | CF₃ | methyl | cyano | methyl | ethyl |
| A-113 | CF₃ | methyl | cyano | ethyl | ethyl |
| A-114 | CF₃ | methyl | cyano | isopropryl | ethyl |
| A-115 | CF₃ | methyl | cyano | methyl | isopropryl |
| A-116 | CF₃ | methyl | cyano | ethyl | isopropryl |
| A-117 | CF₃ | methyl | cyano | isopropryl | isopropryl |
| A-118 | CF₃ | chlorine | bromine | methyl | methyl |
| A-119 | CF₃ | chlorine | bromine | ethyl | methyl |
| A-120 | CF₃ | chlorine | bromine | isopropryl | methyl |
| A-121 | CF₃ | chlorine | bromine | methyl | ethyl |
| A-122 | CF₃ | chlorine | bromine | ethyl | ethyl |
| A-123 | CF₃ | chlorine | bromine | isopropryl | ethyl |
| A-124 | CF₃ | chlorine | bromine | methyl | isopropryl |
| A-125 | CF₃ | chlorine | bromine | ethyl | isopropryl |
| A-126 | CF₃ | chlorine | bromine | isopropryl | isopropryl |
| A-127 | CF₃ | bromine | bromine | methyl | methyl |
| A-128 | CF₃ | bromine | bromine | ethyl | methyl |
| A-129 | CF₃ | bromine | bromine | isopropryl | methyl |
| A-130 | CF₃ | bromine | bromine | methyl | ethyl |
| A-131 | CF₃ | bromine | bromine | ethyl | ethyl |
| A-132 | CF₃ | bromine | bromine | isopropryl | ethyl |
| A-133 | CF₃ | bromine | bromine | methyl | isopropryl |
| A-134 | CF₃ | bromine | bromine | ethyl | isopropryl |
| A-135 | CF₃ | bromine | bromine | isopropryl | isopropryl |
| A-136 | CHF₂ | methyl | chlorine | methyl | methyl |
| A-137 | CHF₂ | methyl | chlorine | ethyl | methyl |
| A-138 | CHF₂ | methyl | chlorine | isopropryl | methyl |
| A-139 | CHF₂ | methyl | chlorine | methyl | ethyl |
| A-140 | CHF₂ | methyl | chlorine | ethyl | ethyl |
| A-141 | CHF₂ | methyl | chlorine | isopropryl | ethyl |
| A-142 | CHF₂ | methyl | chlorine | methyl | isopropryl |
| A-143 | CHF₂ | methyl | chlorine | ethyl | isopropryl |
| A-144 | CHF₂ | methyl | chlorine | isopropryl | isopropryl |
| A-145 | CHF₂ | chlorine | chlorine | methyl | methyl |
| A-146 | CHF₂ | chlorine | chlorine | ethyl | methyl |
| A-147 | CHF₂ | chlorine | chlorine | isopropryl | methyl |
| A-148 | CHF₂ | chlorine | chlorine | methyl | ethyl |
| A-149 | CHF₂ | chlorine | chlorine | ethyl | ethyl |
| A-150 | CHF₂ | chlorine | chlorine | isopropryl | ethyl |
| A-151 | CHF₂ | chlorine | chlorine | methyl | isopropryl |
| A-152 | CHF₂ | chlorine | chlorine | ethyl | isopropryl |
| A-153 | CHF₂ | chlorine | chlorine | isopropryl | isopropryl |
| A-154 | CHF₂ | methyl | cyano | methyl | methyl |
| A-155 | CHF₂ | methyl | cyano | ethyl | methyl |
| A-156 | CHF₂ | methyl | cyano | isopropryl | methyl |
| A-157 | CHF₂ | methyl | cyano | methyl | ethyl |
| A-158 | CHF₂ | methyl | cyano | ethyl | ethyl |
| A-159 | CHF₂ | methyl | cyano | isopropryl | ethyl |
| A-160 | CHF₂ | methyl | cyano | methyl | isopropryl |
| A-161 | CHF₂ | methyl | cyano | ethyl | isopropryl |
| A-162 | CHF₂ | methyl | cyano | isopropryl | isopropryl |
| A-163 | CHF₂ | chlorine | bromine | methyl | methyl |
| A-164 | CHF₂ | chlorine | bromine | ethyl | methyl |
| A-165 | CHF₂ | chlorine | bromine | isopropryl | methyl |
| A-166 | CHF₂ | chlorine | bromine | methyl | ethyl |
| A-167 | CHF₂ | chlorine | bromine | ethyl | ethyl |
| A-168 | CHF₂ | chlorine | bromine | isopropryl | ethyl |
| A-169 | CHF₂ | chlorine | bromine | methyl | isopropryl |
| A-170 | CHF₂ | chlorine | bromine | ethyl | isopropryl |
| A-171 | CHF₂ | chlorine | bromine | isopropryl | isopropryl |
| A-172 | CHF₂ | bromine | bromine | methyl | methyl |
| A-173 | CHF₂ | bromine | bromine | ethyl | methyl |
| A-174 | CHF₂ | bromine | bromine | isopropryl | methyl |
| A-175 | CHF₂ | bromine | bromine | methyl | ethyl |
| A-176 | CHF₂ | bromine | bromine | ethyl | ethyl |
| A-177 | CHF₂ | bromine | bromine | isopropryl | ethyl |
| A-178 | CHF₂ | bromine | bromine | methyl | isopropryl |
| A-179 | CHF₂ | bromine | bromine | ethyl | isopropryl |
| A-180 | CHF₂ | bromine | bromine | isopropryl | isopropryl |
| A-181 | methoxy | methyl | chlorine | methyl | methyl |
| A-182 | methoxy | methyl | chlorine | ethyl | methyl |
| A-183 | methoxy | methyl | chlorine | isopropryl | methyl |
| A-184 | methoxy | methyl | chlorine | methyl | ethyl |
| A-185 | methoxy | methyl | chlorine | ethyl | ethyl |
| A-186 | methoxy | methyl | chlorine | isopropryl | ethyl |
| A-187 | methoxy | methyl | chlorine | methyl | isopropryl |
| A-188 | methoxy | methyl | chlorine | ethyl | isopropryl |
| A-189 | methoxy | methyl | chlorine | isopropryl | isopropryl |
| A-190 | methoxy | chlorine | chlorine | methyl | methyl |
| A-191 | methoxy | chlorine | chlorine | ethyl | methyl |
| A-192 | methoxy | chlorine | chlorine | isopropryl | methyl |
| A-193 | methoxy | chlorine | chlorine | methyl | ethyl |
| A-194 | methoxy | chlorine | chlorine | ethyl | ethyl |
| A-195 | methoxy | chlorine | chlorine | isopropryl | ethyl |
| A-196 | methoxy | chlorine | chlorine | methyl | isopropryl |
| A-197 | methoxy | chlorine | chlorine | ethyl | isopropryl |
| A-198 | methoxy | chlorine | chlorine | isopropryl | isopropryl |
| A-199 | methoxy | methyl | cyano | methyl | methyl |
| A-200 | methoxy | methyl | cyano | ethyl | methyl |
| A-201 | methoxy | methyl | cyano | isopropryl | methyl |
| A-202 | methoxy | methyl | cyano | methyl | ethyl |
| A-203 | methoxy | methyl | cyano | ethyl | ethyl |
| A-204 | methoxy | methyl | cyano | isopropryl | ethyl |
| A-205 | methoxy | methyl | cyano | methyl | isopropryl |
| A-206 | methoxy | methyl | cyano | ethyl | isopropryl |
| A-207 | methoxy | methyl | cyano | isopropryl | isopropryl |
| A-208 | methoxy | chlorine | bromine | methyl | methyl |
| A-209 | methoxy | chlorine | bromine | ethyl | methyl |
| A-210 | methoxy | chlorine | bromine | isopropryl | methyl |
| A-211 | methoxy | chlorine | bromine | methyl | ethyl |
| A-212 | methoxy | chlorine | bromine | ethyl | ethyl |
| A-213 | methoxy | chlorine | bromine | isopropryl | ethyl |
| A-214 | methoxy | chlorine | bromine | methyl | isopropryl |
| A-215 | methoxy | chlorine | bromine | ethyl | isopropryl |
| A-216 | methoxy | chlorine | bromine | isopropryl | isopropryl |
| A-217 | methoxy | bromine | bromine | methyl | methyl |
| A-218 | methoxy | bromine | bromine | ethyl | methyl |
| A-219 | methoxy | bromine | bromine | isopropryl | methyl |
| A-220 | methoxy | bromine | bromine | methyl | ethyl |
| A-221 | methoxy | bromine | bromine | ethyl | ethyl |
| A-222 | methoxy | bromine | bromine | isopropryl | ethyl |
| A-223 | methoxy | bromine | bromine | methyl | isopropryl |
| A-224 | methoxy | bromine | bromine | ethyl | isopropryl |
| A-225 | methoxy | bromine | bromine | isopropryl | isopropryl |

The conversion in step (i) of the process according to the first aspect of the invention for preparing an N-substituted 1H-pyrazole-5-carboxylic acid I-A is a deprotonation of the carbon atom in position 5 of the pyrazole ring of compound II, i.e. an abstraction of a proton in said position. This transformation is effected by contacting a compound II and a base in the presence of a lithium halide. The transformation is, preferably carried out in a solvent. The transformation is preferably carried out under an inert atmosphere, using suitable reaction conditions.

The base used for the reaction in step (i) of the process according to the invention is selected from combinations of a magnesium-organic compound having a carbon bound magnesium and a secondary amine, magnesium amides of secondary amines and mixtures thereof.

For the reaction in step (i) of the inventive process the base is used in an amount that is sufficient to achieve at least 80% deprotonation of the compound of formula (II) in the conversion of step (i) of the process according to the invention. This can typically be accomplished by using the base in an amount, calculated as magnesium, from 1 mol to 2 mol, preferably 1.05 mol to 1.5 mol, more preferably 1.1 mol to 1.4 mol, and in particular 1.15 mol to 1.3 mol, per 1 mol of the compound of formula (II).

In case the base is or includes a combination of a magnesium-organic compound having a carbon bound magnesium and a secondary amine, the magnesium-organic compound is typically selected from C₁-C₈-alkyl magnesium halides, such as methyl magnesium chloride, methyl magnesium bromide, ethyl magnesium chloride, ethyl magnesium bromide, isopropyl magnesium chloride, isopropyl magnesium bromide, butyl magnesium chloride, butyl magnesium bromide, pentyl magnesium chloride, hexyl magnesium bromide, heptyl magnesium chloride or octyl magnesium bromide, C₃-C₈-cycloalkyl magnesium halides, such as cylcohexyl magnesium chloride, cylcohexyl magnesium bromide, cylcopropyl magnesium chloride or cylcopropyl magnesium bromide, and aryl magnesium halides, such as phenyl magnesium bromide or phenyl magnesium chloride. Preferably the magnesium-organic compound is selected from C₁-C₆-alkyl magnesium halides and C₅-C₆-cycloalkyl magnesium halides, more preferably selected from C₁-C₆-alkyl magnesium chlorides, C₁-C₆-alkyl magnesium bromides, C₅-C₆-cycloalkyl magnesium chlorides and C₅-C₆-cycloalkyl magnesium bromides, even more preferably selected from C₁-C₆-alkyl magnesium chlorides, in particular selected from C₁-C₄-alkyl magnesium chlorides, and specifically selected from methyl magnesium chloride, ethyl magnesium chloride, n-propyl magnesium chloride, isopropyl magnesium chloride.

For the purposes of the process of the invention the aforementioned magnesium-organic compounds can also be employed in the form of complexes they from with lithium halides, such as in particular lithium chloride. Examples for such complexes are complexes of C₁-C₆-alkyl magnesium halide with lithiumchloride, in particular C₁-C₆-alkyl magnesium chloride lithium chloride complexes, such as isopropyl magnesium chloride lithium chloride complex and ethyl magnesium chloride lithium chloride complex.

In a preferred embodiment of the present invention the magnesium-organic compound is selected from isopropyl magnesium chloride and isopropyl magnesium chloride lithium chloride complex.

The complex may be preformed in a separate step. The complex may also be prepared in situ by adding the magnesium-organic compound, in particular the C₁-C₆-alkyl magnesium halide, especially the C₁-C₆-alkyl magnesium chloride to the lithium halide, especially to lithium chloride in a suitable solvent, preferably in the presence of the secondary amine and/or the pyrazole compound.

The secondary amine used for the purposes of the process of the present invention is typically selected from:
di-C₁-C₁₄-alkyl amines, preferably di-C₁-C₁₂-alkyl amines, such as dimethylamine, diethylamine, N-methyl-N-ethylamine, N,N-di-n-propylamine, N-methyl-N-isopropyl amine, N-ethyl-N-isopropyl amine, N,N-diisopropylamine, N,N-di-n-butylamine, N,N-di-but-2-ylamine, N,N-di-isobutylamine, N,N-di-tert-butylamine, N,N-dipentylamine, N,N-dihexylamine, N,N-diheptylamine, N-isopropyl-N-but-2-ylamine, N-isopropyl-N-isobutylamine, N-isopropyl-N-tert-butylamine, N,N-bis-(2-methylhexyl)amine, N,N-dioctylamine, N,N-bis-(2-ethylhexyl)amine, N,N-dinonylamine, N,N-bis-(2-propylhexyl)amine, N,N-bis-(2-ethylheptyl)amine, N,N-didecylamine, N,N-bis-(2-propylheptyl)amine, N,N-bis-(2-ethyloctyl)amine, N,N-diundecylamine, N,N-bis-(2-propyloctyl)amine, N,N-didodecylamine, N,N-bis-(2-butyloctyl)amine, and the like;
N—C₁-C₁₄-alkyl N—C₃-C₈-cycloalkyl amines, preferably N—C₁-C₄-alkyl N—C₅-C₇-cycloalkyl amines, such as N-methyl-N-cyclohexylamine, N-ethyl-N-cyclohexylamine and the like;
di-C₃-C₈-cycloalkyl amines, preferably di-C₅-C₇-cycloalkyl amines, such as dicyclopentylamine, N-cyclopentyl-N-cyclohexylamine, dicyclohexylamine, dicycloheptylamine and the like;
saturated 5- to 7-membered heterocyclic amines, which are unsubstituted or substituted by 1, 2, 3 or 4 C₁-C₈-alkyl groups, where the heterocycle in addition to the NH group may contain 1 or 2 further heteroatoms selected from 0 and N, which are in the form of an ether oxygen or in the form of a N—C₁-C₄-alkyl group, preferably saturated 5- to 7-membered heterocyclic amines, which are unsubstituted or substituted by 1 or 2 C₁-C₂-alkyl groups, where the heterocycle in addition to the NH group may contain a further heteroatom selected from 0 and N, which are in the form of an ether oxygen or in the form of a N—C₁-C₂-alkyl group, such as piperidine, 2-methylpiperidine, morpholine, 2-methylmorpholine, 3-methylmorpholine, 1-ethylpiperazine, 1-methylpiperazine, pyrrolidine, 2-methylpyrrolidine, oxazolidine, azepane, 2-methylazepane, 3-methylazepane and the like; and
N-aryl-N—C₁-C₁₄-alkyl amines where the aryl group is unsubstituted or substituted by 1, 2, 3 or 4 radicals selected from C₁-C₄-alkyl and C₁-C₄-alkoxy, preferably N-aryl-N—C₁-C₆-alkyl amines where the aryl group is unsubstituted or substituted by 1 or 2 radicals selected from C₁-C₂-alkyl and C₁-C₂-alkoxy, such as N-methyl-N-phenylamine, N-ethyl-N-phenylamine, N-propyl-N-phenylamine, N-isopropyl-N-phenylamine, N-isobutyl-N-phenylamine, N-tert-butyl-N-phenylamine, N-methyl-N-(4-methylphenyl)amine, N-ethyl-N-(4-methylphenyl)amine, N-isopropyl-N-(4-methylphenyl)amine, N-tert-butyl-N-(4-methylphenyl)amine, N-methyl-N-(3-methylphenyl)amine, N-ethyl-N-(3-methylphenyl)amine, N-propyl-N-(3-methylphenyl)amine, N-isobutyl-N-(3-methylphenyl)amine, N-methyl-N-(4-methoxyphenyl)amine, N-ethyl-N-(4-methoxyphenyl)amine, N-isopropyl-N-(4-methoxyphenyl)amine, N-tert-butyl-N-(4-methoxyphenyl)amine, N-methyl-N-(3-methoxyphenyl)amine, N-ethyl-N-(3-methoxyphenyl)amine, N-propyl-N-(3-methoxyphenyl)amine, N-isobutyl-N-(3-methoxyphenyl)amine, N-methyl-N-(4-ethylphenyl)amine, N-ethyl-N-(4-ethylphenyl)amine, N-isopropyl-N-(4-ethylphenyl)amine, N-methyl-N-(3-ethylphenyl)amine, N-ethyl-N-(3-ethylphenyl)amine, N-tert-butyl-N-(3-ethylphenyl)amine, N-methyl-N-(4-ethoxyphenyl)amine, N-ethyl-N-(4-ethoxyphenyl)amine, N-isopropyl-N-(4-ethoxyphenyl)amine, N-tert-butyl-N-(4-ethoxyphenyl)amine, N-isopropyl-N-(3-ethoxyphenyl)amine, N-tert-butyl-N-(3-ethoxyphenyl)amine, N-methyl-N-(3,5-dimethylphenyl)amine, N-ethyl-N-(3,5-dimethylphenyl)amine, N-isopropyl-N-(3,5-dimethylphenyl)amine, N-tert-butyl-N-(3,5-dimethylphenyl)amine, N-methyl-N-(3-methyl-5-methoxyphenyl)amine, N-ethyl-N-(3-methyl-5-methoxyphenyl)amine, N-propyl-N-(3-methyl-5-methoxyphenyl)amine, N-isobutyl-N-(3-methyl-5-methoxyphenyl)amine, N-methyl-N-(3,5-dimethoxyphenyl)amine, N-ethyl-N-(3,5-dimethoxyphenyl)amine, N-isopropyl-N-(3,5-dimethoxyphenyl)amine, N-tert-butyl-N-(3,5-dimethoxyphenyl)amine and the like.

In a preferred embodiment of the present invention the secondary amine is a compound of the formula (RN)

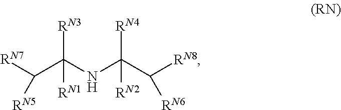

wherein R^{N1}, R^{N2}, R^{N3}, R^{N4}, R^{N5}, R^{N6}, R^{N7}, R^{N8}, independently of each other are selected from the group of hydrogen and C₁-C₈-alkyl, where the two radicals R^{N3} and R^{N4} may also form a (CH₂)ₚ group with p being 2, 3 or 4, wherein one CH₂ group may be replaced by an oxygen atom or a group N—C₁-C₄-alkyl;

provided that the total number of carbon atom in the radicals R^{N1}, R^{N2}, R^{N3}, R^{N4}, R^{N5}, R^{N6}, R^{N7} and R^{N8} is from 2 to 24.

Preference is given to the secondary amines of the formula (RN) according to the aforementioned embodiment of the invention, wherein R^{N1} and R^{N2} independently of each other are hydrogen or methyl, R^{N3} and R^{N4} independently of each other are C₁-C₄-alkyl, in particular C₁-C₂-alkyl, and R^{N5}, R^{N6}, R^{N7} and R^{N8} are hydrogen, such as N,N-diisopropylamine, N,N-di-tert-butylamine, N-isopropyl-N-tert-butylamine, N,N-di-but-2-ylamine, N-isopropyl-N-but-2-ylamine, N-tert-butyl-N-but-2-ylamine, N,N-di-pent-2-ylamine, N,N-di-(2,2-dimethylpropyl)amine and the like.

Preference is also given to the secondary amines of the formula (RN) according to the aforementioned embodiment of the invention, wherein R^{N1} and R^{N2} independently of each other are hydrogen or methyl, in particular methyl, R^{N3} and R^{N4} form a (CH₂)ₚ group with p being 2, 3 or 4, wherein one CH₂ group in (CH₂)ₚ may be replaced by an oxygen atom or a group N—C₁-C₄-alkyl, in particular N—C₁-C₂-alkyl and specifically N-methyl, and R^{N5}, R^{N6}, R^{N7} and R^{N8} are hydrogen, such as 2,2,6,6-tetramethylpiperidine, 2,2,4,6,6-pentamethylpiperazine, 3,3,5,5-tetramethylmorpholine, 2,2,5,5-tetramethylpyrrolidine, 2,2,7,7-tetramethylazepane and the like.

Preference is further given to the secondary amines of the formula (RN) according to the aforementioned embodiment of the invention, wherein R^{N1} and R^{N2} independently of each other are hydrogen or methyl, in particular hydrogen, R^{N3} and R^{N4} are hydrogen and R^{N5}, R^{N6}, R^{N7} and R^{N8} independently of each other are C₁-C₈-alkyl. In this context those secondary amines RN are particular preferred wherein R^{N1} and R^{N2} are both hydrogen and R^{N5} and R^{N6} independently of each other are C₁-C₅-alkyl, in particular C₂-C₄-alkyl, and R^{N7} and R^{N8} independently of each other are C₄-C₈-alkyl, in particular C₆-C₈-alkyl, such as N,N-bis-(2-ethylhexyl)amine, N,N-bis-(2-propylhexyl)amine, N-(2-ethylhexyl)-N-(2-propylheptyl)amine, N,N-bis-(2-ethylheptyl)amine, N,N-bis-(2-propylheptyl)amine, N,N-bis-(2-ethyloctyl)amine, N,N-bis-(2-butylheptyl)amine, N,N-bis-(2-propyloctyl)amine, N-(2-propylheptyl)-N-(2-butyloctyl)-amine, N,N-bis-(2-butyloctyl)amine and the like.

In another preferred embodiment of the present invention the secondary amine is a compound of the formula (RN′)

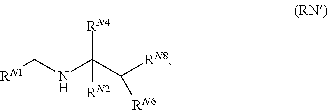

wherein R^{N1}, R^{N2}, R^{N4}, R^{N6}, R^{N8}, independently of each other are selected from the group of hydrogen and C₁-C₈-alkyl, where the two radicals R^{N2} and R^{N6} may also form a (CH₂)_{q} group with q being 1, 2, 3 or 4, wherein one CH₂ group may be replaced by an oxygen atom or a group N—C₁-C₄-alkyl;

provided that the total number of carbon atom in the radicals R^{N1}, R^{N2}, R^{N4}, R^{N5}, R^{N6} and R^{N8} is from 1 to 16.

Preference is given to the secondary amines of the formula (RN′) according to the aforementioned embodiment of the invention, wherein R^{N1} and R^{N2} independently of each other are hydrogen or methyl, R^{N4} is C₁-C₄-alkyl, in particular C₁-C₂-alkyl, and R^{N6} and R^{N8} are hydrogen, such as N-methyl-N-isopropylamine, N-methyl-N-2-butylamine, N-methyl-N-tert-butylamine, N-ethyl-N-isopropylamine, N-ethyl-N-2-butylamine, N-ethyl-N-tert-butylamine, and the like.

The secondary amine used for the purposes of the process of the present invention is preferably selected from the group consisting of dimethylamine, diethylamine, N-methyl-N-ethylamine, N-methyl-N-isopropyl amine, N-ethyl-N-isopropyl amine, N-methyl-N-cyclohexyl amine, N-ethyl-N-cyclohexyl amine, dicyclohexylamine, piperidine, 2-methylpiperidine, morpholine, 1-methylpiperazine, pyrrolidine, oxazolidine, 2,2,6,6-tetramethylpiperidine, 2,2,4,6,6-pentamethylpiperazine, 3,3,5,5-tetramethylmorpholine, 2,2,5,5-tetramethylpyrrolidine, N,N-diisopropylamine, N-isopropyl-N-tert-butylamine, N,N-di-tert-butylamine, N,N-di-but-2-ylamine, N,N-bis-(2-ethylhexyl)amine, N-(2-ethylhexyl)-N-(2-propylheptyl)amine, N,N-bis-(2-propylheptyl)amine, N,N-bis-(2-butyloctyl)amine, N-methyl-N-phenylamine, N-ethyl-N-phenylamine, N-methyl-N-(4-methylphenyl)amine and N-methyl-N-(4-methoxyphenyl)amine, in particular selected from the group consisting of dimethylamine, diethylamine, dicyclohexylamine, piperidine, morpholine, 2,2,6,6-tetramethylpiperidine, 2,2,4,6,6-pentamethylpiperazine, 3,3,5,5-tetramethylmorpholine, N,N-diisopropylamine, N-isopropyl-N-tert-butylamine, N,N-di-tert-butylamine, N,N-bis-(2-ethylhexyl)amine, N,N-bis-(2-propylheptyl)amine, N,N-bis-(2-butyloctyl)amine and N-methyl-N-phenylamine, and specifically selected from the group consisting of dimethylamine, dicyclohexylamine, N,N-diisopropylamine, N-isopropyl-N-tert-butylamine, 2,2,6,6-tetramethylpiperidine and N,N-bis-(2-ethylhexyl)amine.

In a special embodiment of the invention, the secondary amine used for the purposes of the process of the present invention is N-methyl-N-isopropyl amine.

In case the base is or includes a combination of a magnesium-organic compound having a carbon bound magnesium and a secondary amine, the secondary amine is employed in an amount of from 0.01 mol to 2 mol per 1 mol of the compound of formula (II). According to a preferred embodiment of the present invention the secondary amine is employed either in a catalytic amount of from 0.01 to 0.5, more preferably 0.03 to 0.3 and in particular 0.05 to 0.25 mol per 1 mol of the compound II. According to another preferred embodiment the secondary amine is employed in a stoichiometric or near stoichiometric amount of from 0.8 to 2, more preferably 1.0 to 1.35 and in particular 1.1 to 1.3 mol per 1 mol of the compound II.

In a particular embodiment the base used for the conversion in step (i) of the process according to the present invention is a combination of a magnesium organic compound selected from the aforementioned alkyl and cycloalkyl magnesium halides, in particular those mentioned as preferred, with a secondary amine, in particular a secondary amine mentioned herein as preferred. As discussed before, the magnesium organic compounds may be employed as is or in the form of the complexes they form with lithium halides, in particular lithium chloride, such as isopropyl magnesium chloride lithium chloride complex or ethyl magnesium chloride lithium chloride complex.

The conversion in step (i) of the process according to the present invention is performed in the presence of lithium halide, which is preferably lithium chloride. The lithium halide is typically used in an amount from 0.05 to 2.5 mol, preferably from 0.1 to 2 mol, more preferably from 0.5 to 1.5 mol, in particular from 1.0 to 1.4 mol and especially from 1.1 to 1.3 mol, in each case per mol of magnesium in the base. The lithium halide is usually added separately to the reaction mixture or in the form of its complex with either a magnesium-organic compound having a carbon bound or a magnesium amide of secondary amine, depending on whether the base used in the step (i) is a combination of a magnesium-organic compound and a secondary amine or a magnesium amide. According to a preferred embodiment of the invention the lithium halide, in particular lithium chloride, is added to the reaction mixture in the form of one of its aforementioned complexes.

In case the base is or includes a combination of a magnesium-organic compound having a carbon bound magnesium and a secondary amine, it is possible to prepare the magnesium-organic compound in situ. E.g. if the magnesium-organic compound is a Grignard compound, such as an alkyl magnesium halide or a cycloalkyl magnesium halide, the Grignard compound may be prepared in the reactor by reacting magnesium or a magnesium containing alloy with an alkyl halide or cycloalkyl halide and then add the lithium halide and the secondary amine.

Without being bound to theory it is assumed that in case the base used in step (i) of the inventive process is a combination of a magnesium-organic compound and a secondary amine these two compound react in-situ to give the corresponding magnesium amide, which then effects the deprotonation of compound II.

In case the base used in step (i) of the process according to the invention is or includes a magnesium amide of a secondary amine, the magnesium amide is generally obtainable by reacting a magnesium-organic compound having a carbon bound magnesium and a secondary amine. Preferred magnesium-organic compounds and secondary amines in this regard are those mentioned herein, in particular those mentioned as preferred.

Accordingly, magnesium amides that are preferred in the context of the present invention are selected from dimethylamido magnesium chloride, diethylamido magnesium chloride, N-methyl-N-ethylamido magnesium chloride, dicyclohexylamido magnesium chloride, 1-piperidinyl magnesium chloride, 2-methylpiperidin-1-yl magnesium chloride, 4-morpholinyl magnesium chloride, 4-methylpiperazin-1-yl magnesium chloride, 1-pyrrolidinyl magnesium chloride, 3-oxazolidinyl magnesium chloride, 2,2,6,6-tetramethylpiperidin-1-yl magnesium chloride, 2,2,4,6,6-pentamethylpiperazin-1-yl magnesium chloride, 3,3,5,5-tetramethylmorpholin-4-yl magnesium chloride, 2,2,5,5-tetramethylpyrrolidin-1-yl magnesium chloride, N,N-diisopropylamido magnesium chloride, N-isopropyl-N-tert-butylamido magnesium chloride, N,N-di-tert-butylamido magnesium chloride, N,N-di-but-2-ylamido magnesium chloride, N,N-bis-(2-ethylhexyl)amido magnesium chloride, N-(2-ethylhexyl)-N-(2-propylheptyl)amido magnesium chloride, N,N-bis-(2-propylheptyl)amido magnesium chloride, N,N-bis-(2-butyloctyl)amido magnesium chloride, N-methyl-N-phenylamido magnesium chloride, N-ethyl-N-phenylamido magnesium chloride, N-methyl-N-(4-methylphenyl)amido magnesium chloride and N-methyl-N-(4-methoxyphenyl)amido magnesium chloride, and in particular selected from the group consisting of dimethylamido magnesium chloride, diethylamido magnesium chloride, dicyclohexylamido magnesium chloride, 1-piperidinyl magnesium chloride, 4-morpholinyl magnesium chloride, 2,2,6,6-tetramethylpiperidin-1-yl magnesium chloride, 2,2,4,6,6-pentamethylpiperazin-1-yl magnesium chloride, 3,3,5,5-tetramethylmorpholin-4-yl magnesium chloride, N,N-diisopropylamido magnesium chloride, N-isopropyl-N-tert-butylamido magnesium chloride, N,N-di-tert-butylamido magnesium chloride, N,N-bis-(2-ethylhexyl)amido magnesium chloride, N,N-bis-(2-propylheptyl)amido magnesium chloride, N,N-bis-(2-butyloctyl)amido magnesium chloride and N-methyl-N-phenylamido magnesium chloride.

Magnesium amides that are likewise preferred in the context of the present invention are selected from N-methyl-N-isopropylamido magnesium chloride, N-methyl-N-2-butylamido magnesium chloride, N-methyl-N-tert-butylamido magnesium chloride, N-ethyl-N-isopropylamido magnesium chloride, N-ethyl-N-2-butylamido magnesium chloride and N-ethyl-N-tert-butylamido magnesium chloride, especially N-methyl-N-isopropylamido magnesium chloride.

The magnesium amides of secondary amines may be employed in step (i) of the inventive process as is or in the form of the complexes they form with lithium halides, in particular lithium chloride, such as N,N-diisopropylamido magnesium chloride lithium chloride complex, dicyclohexylamido magnesium chloride lithium chloride complex, N-methyl-N-isopropylamido magnesium chloride lithium chloride complex, N-ethyl-N-isopropylamido magnesium chloride lithium chloride complex, or 2,2,6,6-tetramethylpiperidin-1-yl magnesium chloride lithium chloride complex.

In a preferred embodiment of the present invention the magnesium amides of secondary amines are selected from N,N-diisopropylamido magnesium chloride, dicyclohexylamido magnesium chloride, 2,2,6,6-tetramethylpiperidin-1-yl magnesium chloride, N,N-diisopropylamido magnesium chloride lithium chloride complex, dicyclohexylamido magnesium chloride lithium chloride complex and 2,2,6,6-tetramethylpiperidin-1-yl magnesium chloride lithium chloride complex.

In a particular preferred embodiment of the present invention the base used in step (i) of the inventive process is selected from combinations of a magnesium-organic compound having a carbon bound magnesium and a secondary amine, preferably those combinations mentioned herein as preferred, and in particular is selected from combinations of isopropyl magnesium chloride or the lithium chloride complex thereof with a secondary amine selected from dimethylamine, dicyclohexylamine, N,N-diisopropylamine, N-isopropyl-N-tert-butylamine, 2,2,6,6-tetramethylpiperidine and N,N-bis-(2-ethylhexyl)amine.

In a special embodiment of the invention, the base used in step (i) of the inventive process is a combination of isopropyl magnesium chloride or the lithium chloride complex thereof with N-methyl-N-isopropyl amine.

The conversion of step (i) is usually performed in an aprotic organic solvent or a mixture of aprotic organic solvents. Suitable aprotic organic solvents here include, for example, aprotic solvent having an ether moiety, e.g. aliphatic and cycloaliphatic C₃-C₈ ethers, in particular aliphatic C₃-C₆ ethers such as dimethoxyethane, diethylene glycol dimethyl ether, diethyl ether, dipropyl ether, diisopropyl ether, di-n-butyl ether, methyl isobutyl ether, methyl cyclopentyl ether, tert-butyl methyl ether and tert-butyl ethyl ether, alicyclic C₃-C₆ ethers, such as tetrahydrofuran (THF), tetrahydropyran, 2-methyltetrahydrofuran, 3-methyltetrahydrofuran and dioxane, aliphatic hydrocarbons, such as pentane, hexane, heptane and octane, and also petroleum ether, cycloaliphatic hydrocarbons, such as cyclopentane and cyclohexane, aromatic hydrocarbons, such as benzene, toluene, the xylenes and mesitylene, or mixtures of these solvents with one another.

The solvent for the conversion in step (i) preferably comprises at least one aprotic solvent having an ether moiety, which is in particular selected from aliphatic and alicyclic ethers, especially form C₃-C₆-aliphatic ethers and C₄-C₆ alicyclic ethers, or a mixture thereof. The solvent for the conversion in step (i) is in particular selected from aprotic solvents having an ether moiety, which is in particular selected from aliphatic and alicyclic ethers, especially form C₃-C₆-aliphatic ethers and C₄-C₆ alicyclic ethers, or a mixture thereof. Preferably, THF or dimethoxyethane or solvent mixtures comprising them are used as solvent. In a particular embodiment THF is used as solvent. In another particular embodiment, a mixture of THF and dimethoxyethane is used as a solvent. If compound II is initially present in the reaction vessel in a solvent, which is preferably THF, the base, or components thereof, may be added in the same solvent or a different solvent, selected from diethylether and dimethoxyethane.

The solvent may contain an aprotic amide or urea as a cosolvent, e.g. N-methyl pyrrolidone, N,N-dimethyl acetamide, N,N′-dimethyl propylene urea (DMPU), N,N,N′,N′-Tetramethyl urea etc.

The total amount of the solvent used in step (i) of the process according to the invention is typically in the range from 1000 to 10000 g, preferably in the range from 2000 to 9000 g, in particular from 3000 to 8000 g, based on 1 mol of the compound II.

Preference is given to using solvents which are essentially anhydrous, i.e. have a water content of less than 5000 ppm, in particular less than 2000 ppm, especially less than 1000 ppm. Generally, the water contained in the solvent will react with the magnesium organic compound or the magnesium amide resulting in a certain loss of base, which can be compensated by using higher amounts of the magnesium organic compound or the magnesium amide.

In general, the reaction of step (i) is performed under temperature control.

The reaction of step (i) may be performed in any type of reactor, e.g. a reaction vessel, which is operated continuously or batch-wise, or a continuously operated tube like reaction zone. The reaction vessel may be a closed or unclosed reaction vessel, optionally with stirring and/or a cooling device. The tube like reaction zone may have static or dynamic mixers. The reactor may also be a micro-reactor.

A suitable temperature profile for the reaction in step (i) is determined by several factors, for example the reactivity of the compound II used and the type of base selected, the type of solvent or co-solvent, if present, and can be determined by the person skilled in the art in the individual case, for example by simple preliminary tests. Generally the deprotonation of step (i) will be performed at a temperature in the range from −30 to +50° C., in particular from −20 to +20° C., most preferably under cooling from −10 to +10° C.

The reactants can in principle be contacted with one another in any desired sequence. For example, the compound II, optionally dissolved in a solvent or in dispersed form can be initially charged and then the base, optionally in dissolved or dispersed form, is added, or, conversely, the base, optionally dissolved or dispersed in a solvent can be initially charged and admixed with the compound II. Alternatively, the two reactants can also be fed simultaneously to the reactor. In case a combination of a magnesium-organic compound having a carbon bound magnesium and a secondary amine is used as base, prior to contacting with compound II the magnesium-organic compound and the secondary amine may be reacted with each other for a period of time at a temperature of typically from −20 to +20° C., preferably in a solvent that in general is selected from the aprotic solvents mentioned above, in particular from those mentioned as preferred. Alternatively, in particular if the secondary amine is employed in a catalytic amount, the secondary amine is mixed with compound II optionally together with a solvent to form a solution or dispersion and afterwards the obtained mixture is contacted with the magnesium-organic compound.

It has been found to be appropriate to initially charge the compound II, preferably in a solvent, and then adjust the reaction mixture to a temperature in the range of −20 to 50° C., preferably in the range of −10 to 15° C., depending on the reaction conditions of the individual case and in particular depending on the specific base to be used. In case a combination of a magnesium-organic compound having a carbon bound magnesium and a secondary amine is used as base, the base is separately prepared by reacting the magnesium-organic compound and the secondary amine for a period of time preferably in a solvent at a temperature of typically from −20 to +20° C. Subsequently the base, optionally in a solvent, is added either stepwise, continuously or in one portion and the reaction is allowed to continue for a period of time, possibly at the same temperature, at an elevated temperature or at a gradually rising temperature, wherein the upper limit of the temperature is the upper limit of the temperature ranges described above as preferred.

In case a combination of a magnesium-organic compound and a secondary amine is used as base and the secondary amine is employed in a catalytic amount, it has also been found to be appropriate to initially mix the compound II and the secondary amine, preferably in a solvent, and then adjust the reaction mixture to a temperature in the range of −20 to 50° C., preferably in the range of −10 to 15° C., depending on the reaction conditions of the individual case and in particular depending on the specific base to be used. Afterwards the magnesium-organic compound, optionally in a solvent, is added either stepwise, continuously or in one portion and the reaction is allowed to continue for a period of time, possibly at the same temperature, at an elevated temperature or at a gradually rising temperature, wherein the upper limit of the temperature is the upper limit of the temperature ranges described above as preferred.

For the conversion in step (i), compound II and the base are brought into contact at a set temperature typically in the range of −30 to 50° C., preferably of −20 to 30° C. and in particular of −10 to 15° C. Afterwards the conversion is usually continued either at the set temperature or by applying a temperature gradient with the set temperature as the lower limit and an upper limit in the range of −20 to 35°, preferably of −15 to 30° C. and in particular of −10 to 25° C. or ambient temperature. Ambient temperature is to be understood from 15 to 28° C., preferably from 20 to 25° C.

The reaction product obtained from the conversion in step (i) of the inventive process is usually subjected without preceding work-up to the conversion in step (ii) of the process according to the first aspect of the invention. To this end, typically the reaction mixture obtained after the completion of the conversion in step (i) is directly introduced to the conversion in step (ii).

The conversion in step (ii) of the process according to the first aspect of the invention for preparing an N-substituted 1H-pyrazole-5-carboxylic acid of the formula (I-A) is a carboxylation of the intermediate product obtained in step (i) of the process. This conversion comprises an electrophilic attack of the carbon atom present in carbon dioxide on the deprotonated carbon atom in position 5 of the pyrazole ring of the intermediate derived from compound II. Said electrophilic attack results in the covalent attachment of the carboxylate group CO₂⁻ and, as a consequence, in the formation of the magnesium salt of the N-substituted 1H-pyrazole-5-carboxylic acid I-A. This reaction is effected by contacting the intermediate obtained in step (i) with carbon dioxide or a carbon dioxide equivalent, preferably in a solvent and under an inert atmosphere, using suitable reaction conditions. The obtained magnesium salt of the compound I-A can optionally be converted into the free acid of the formula (I-A) by an aqueous workup.

Suitable carbon dioxide equivalents for the carboxylation in step (ii) are compounds which react in the same manner as carbon dioxide or which have a capability to release carbon dioxide. These carbon dioxide equivalents may be used instead of carbon dioxide itself, provided they are free from water, to avoid side reactions. However, carbon dioxide is preferred as carboxylation reagent in step (ii).

The reactants can in principle be contacted with one another in any desired sequence. For example, the reaction mixture obtained from step (i) that includes the intermediate product resulting from the deprotonation in step (i), optionally mixed with additional solvent, can be initially charged and then solid or gaseous carbon dioxide, optionally in dissolved form, is added, or bubbled through the reaction mixture, or, alternatively, the atmosphere of the reaction vessel is exchanged to carbon dioxide bringing the reaction mixture into contact by suitably stirring. It is also possible to charge a solution or solid carbon dioxide into the reactor and then feed the intermediate product resulting from the deprotonation in step (i), preferably as a solution, to the reactor.

In case the reaction mixture of step (i) is admixed with additional solvent before the carboxylation in step (ii) is initiated, said additional solvent is an aprotic solvent which in particular is selected from the aprotic organic solvents mentioned herein before, especially from those mentioned as preferred. Preferably, the additional solvent is essentially anhydrous, i.e. it has a water content of less than 2000 ppm, in particular less than 1000 ppm.

Frequently, the carbon dioxide or carbon dioxide equivalent is introduced into the reaction of step (ii) in gaseous form either by bubbling through the reaction mixture or by changing the atmosphere to carbon dioxide with simultaneous vigorous stirring, or dissolved in a suitable solvent that is generally selected from the apolar aprotic organic solvents mentioned before. In other embodiments carbon dioxide is introduced into the reaction of step (ii) in solid form, i.e. by adding solid carbon dioxide to the reaction mixture, preferably with simultaneous vigorous stirring.

According to a particular embodiment of the invention, the carboxylation in step (ii) is effected by bubbling gaseous carbon dioxide through the reaction solution. Preferably, the gaseous carbon dioxide is dry, i.e. free from water. The pressure of the carbon dioxide gas is from 0.9 to 20 bar, preferred from 0.9 to 10 bar, more preferred from 0.95 to 2 bar, most preferred from 0.95 to 1.1 bar.

The progress of step (ii) of the reaction depends on the consumption of carbon dioxide, which in general is used in excess. The determination of the end of this reaction is usually done by monitoring the reaction enthalpy. Once, the exothermic reaction has ceased, the conversion to the magnesium salt of the acid of formula (I-A) is complete and no more carbon dioxide needs to be introduced to the reaction mixture. The determination of the end of this reaction may also be monitored by analytical chromatography, e.g. by thin layer chromatography or by HPLC.

In general, the conversion in step (ii) is performed under temperature control.

The reaction of step (ii) may be performed in any type of reactor, e.g. a reaction vessel, which is operated continuously or batch-wise, or a continuously operated tube like reaction zone. The reaction vessel may be a closed or unclosed reaction vessel, optionally with stirring and/or a cooling device. The tube like reaction zone may have static or dynamic mixers. The reactor may also be a micro-reactor.

A suitable temperature profile for the reaction in step (ii) is determined by several factors, in particular the type of base that was used in the deprotonation of step (i), the reactivity of the intermediate obtained in step (i) and the carboxylation reagent selected, and can be determined by the person skilled in the art for each individual case by conventional measures, such as preliminary tests. Generally the reaction will be performed at temperatures ranging from −40 to +80° C., in particular from −20 to +50° C.

Frequently, the reaction mixture obtained after completion of step (i) is adjusted to a temperature in the range of −30 to +60° C., preferably in the range of −20 to +50° C., if required, and then the carboxylation reagent, optionally dissolved in a solvent or in gaseous form, is added. The reaction is allowed to continue for a period of time, possibly at the same temperature, or alternatively at an elevated or gradually rising temperature. Preferably, the temperature is controlled by the speed of the addition of the carboxylation reagent: As the reaction temperature will mostly rise during the reaction, a higher speed will increase the temperature of the reaction mixture. The speed of the carboxylation reagent addition is adjusted in a manner that the temperature of the reaction mixture is kept at the optimum where the reaction proceeds whereas side reactions are avoided.

The intermediate from step (i) and the reagent are brought into contact in step (ii) at a set temperature typically in the range of −30 to +60° C., preferably of −20 to +50° C. and in particular of −10 to +45° C. or ambient temperature. Afterwards the conversion is usually continued either at the set temperature or by applying a temperature gradient with the set temperature as the lower limit and an upper limit in the range of −10 to +60° C., preferably of −5 to +50° C. and in particular of 0 to +50° C. or ambient temperature, and then optionally allow the reaction to proceed at the upper limit temperature.

The reaction mixture obtained after the conversion in step (ii) containing the magnesium salt of the N-substituted 1H-pyrazole-5-carboxylic acid of the formula (I-A) as product, can be employed without purification in the next step or can be subjected to a workup procedure before introducing it to a subsequent reaction step. It is also possible to change the solvent for the next reaction step, even in the case of absence of a purification step. In a particular embodiment, the solvent used in the previous step (ii) is at least partly removed, and, in preparation for the next step, the crude reaction mixture is dissolved in a different solvent, preferably an aliphatic, cycloaliphatic or aromatic hydrocarbon, which may be chlorinated, e.g. dichloromethane, dichloroethane, hexane, cyclohexane, chlorobenzene or toluene, or in an ester solvent, such as a C₁-C₆-alkyl ester of an aliphatic C₁-C₄-carboxylic ester, in particular a C₁-C₆-alkyl ester of acetic acid or propionic acid such as ethyl acetate, propyl acetate, butyl acetate or ethyl propionate, or in a mixture thereof. In another particular embodiment, the solvent of the previous step (ii) is not removed but the reaction mixture, optionally after washing and/or filtration is directly employed in the subsequent step.

The magnesium salt of the N-substituted 1H-pyrazole-5-carboxylic acid I-A can be converted to the corresponding acid chloride (N-substituted 1H-pyrazole-5-carbonyl chloride compound of the formula (I) either directly or via the free acid I-A which is obtainable in step (ii) by an optional aqueous workup of the magnesium salt of the acid I-A.

Accordingly the process according to the second aspect of the invention for preparing a N-substituted 1H-pyrazole-5-carbonyl chloride of the formula (I) comprises the steps of:
a) preparing an N-substituted 1H-pyrazole-5-carboxylic acid of the formula (I-A) or a magnesium salt thereof by the process described herein above, and
b) converting the compound of the formula (I-A) or its magnesium salt into the compound of formula (I) by treatment with a chlorinating agent.

The direct conversion of the magnesium salt of the acid (I-A) to the acid chloride I via chlorination in step (b) is effected in analogy to the methods known in the art for the preparation of acid chlorides from the corresponding acids, by reacting the magnesium salt of the acid I-A) with a chlorinating agent, e.g. thionyl chloride, phosphorous pentachloride, phosphorous trichloride or oxalyl chloride, optionally in the presence of catalytic amounts of a polar carboxamide such as N,N-dimethylformamide (DMF). For example, U.S. Pat. No. 4,544,654 describes a conversion of a sodium salt of a carboxylic acid to the corresponding acid chloride, which method can be applied here by analogy. The chlorination of the magnesium salt of the acid I-A is preferably effected in a non-polar solvent, e.g. an aliphatic cycloaliphatic or aromatic hydrocarbon, which may be chlorinated, e.g. dichloromethane, dichloroethane, hexane, cyclohexane, chlorobenzene or toluene. The chlorination of the magnesium salt of the acid I-A may also be effected in the solvent used for deprotonation/carboxylation or in a mixture of these solvents with the aforementioned non-polar solvents. The chlorination of the magnesium salt of the acid I-A is generally effected at a temperature from −5° C. to +140° C., or from 0 to 110° C., or preferably from 0 to 100° C. The chlorination of the magnesium salt of the acid I-A is preferably effected from 0 to 25° C. using oxalyl chloride or from 20 to 110° C. using thionyl chloride.

The conversion of the magnesium salt of the N-substituted 1H-pyrazole-5-carboxylic acid of the formula (I-A) to the corresponding free acid (I-A) is effected by aqueous workup, in particular by an aqueous acidification of the reaction solution of step (ii), e.g. by addition of aqueous acids, such as hydrochloric acid, sulfuric acid, phosphoric acid or the like. The resulting acid compound I-A can be isolated or employed in the next reaction step without purification. Preferably, the acid compound I-A is purified at least by a workup in aqueous media and isolated from the organic phase after drying.

The conversion of the N-substituted 1H-pyrazole-5-carboxylic acid of the formula (I-A) to the corresponding acid chloride (N-substituted 1H-pyrazole-5-carbonyl chloride compound of the formula (I)) in step (b) is effected by standard methods of preparation of acid chlorides, as for example described in Organikum, Wiley-VCH, Weinheim, 21st ed. 2001, p. 498, e.g. by reacting I-A with a chlorinating agent, e.g. thionyl chloride or oxalyl chloride, optionally in the presence of catalytic amounts of a polar carboxamide such as DMF. The chlorination of the free acid I-A is preferably effected in an non-polar solvent, e.g. an aliphatic cycloaliphatic or aromatic hydrocarbon, which may be chlorinated, e.g. dichloromethane, dichloroethane, hexane, cyclohexane, chlorobenzene or toluene and especially in toluene. The chlorination of the free acid I-A is preferably effected at a temperature from −5° C. to +140° C. or from 0 to 110° C., in particular from 0 to 25° C. using oxalyl chloride or from 20 to 110° C. using thionyl chloride.

The reaction mixture obtained after the chlorination in step (b) of the aforementioned inventive process, which contains the N-substituted 1H-pyrazole-5-carbonyl chloride compound of the formula (I) as product, may be subjected to a workup procedure before introducing it to a subsequent reaction step. However, it is also possible to use the crude reaction mixture obtained from the reaction of I-A or its magnesium salt with the chlorinating agent, optionally after filtration. The workup is typically effected by non-aqueous means known in the art to be applicable for similar reactions. Preferably, the reaction mixture, optionally after mixing it with an non-polar aprotic solvent, that usually is an aliphatic ether, an acyclic ether, an aliphatic or cycloaliphatic hydrocarbon, aromatic hydrocarbon or a mixture of the aforementioned solvents, in particular cyclohexane or toluene and specifically toluene, is worked-up by filtering off solids that may be present. The filtered solids, if present, are washed with the solvent, the combined filtrate is concentrated by evaporation and the residue is extracted with a non-polar aprotic solvent that typically is the same as used before. Undissolved solids may be again filtered off, washed with the solvent and the product is isolated from the resulting filtrate, e.g. by removing solvents via evaporation or distillation or by inducing crystallization, optionally after concentration of the filtrate. The raw N-substituted 1H-pyrazole-5-carbonyl chloride compound I thus obtained can be used directly in step (c) of the process according to the third aspect of the invention or sent to other uses. Alternatively, it can be retained for a later use or further purified beforehand. For further purification, it is possible to use one or more methods known to those skilled in the art, for example recrystallization, distillation, sublimation, zone melting, melt crystallization or chromatography. It is however preferred to subject compound I to a subsequent synthetic step in the form of the raw material obtained directly after the workup procedure.

The compounds of formula (I) are known e.g. from WO 2003/015519 or WO 2003/106427 or they can be prepared by analogy to the methods described therein or in WO 2008/126858, WO 2008/126933, WO 2008/130021, WO 2007/043677 and Bioorganic and Medicinal Chemistry Letters 2005, 15, 4898-4906.

In step (c) of the process according to the third aspect of the invention for preparing a sulfimine compound of formula (VI), a compound of formula (VII) is reacted with a pyrazole compound of formula (I) to yield a compound of formula (VI). The reaction can be carried out by analogy to conventional amidation reactions of carboxylic acid chlorides with aromatic amines as described e.g. in WO 2003/015519, WO 2006/062978, WO 2008/07158 or WO 2009/111553. Surprisingly, the group N═S(O)ₜR⁶R⁷ does not interfere with the amidation reaction. Rather, the compounds of formula (VI), can be obtained in high yields with high purity.

Usually, the compounds of formula (VII) and the compounds of formula (I) are preferably employed in stoichiometric or almost stoichiometric amount. Generally, the relative molar ratio of the compounds of formula (VII) to the compounds of formula (I) will be in a range from 1.1:1 to 1:2, preferably from 1.1:1 to 1:1.2 and in particular from 1.05:1 to 1:1.1.

It has been found advantageous to carry out step (c) in the presence of a base. Suitable bases include bases which are soluble or insoluble in the reaction medium. The base may be used in catalytic or stoichiometric amounts. The amount of base may preferably be in the range from 0.5 to 2 mol, in particular from 0.75 to 1.5 mol per mol of compound I.

Suitable bases include but are not limited to oxo bases and amine bases. Suitable oxo bases include but are not limited to carbonates, in particular alkali metal carbonates, such as lithium, sodium or potassium carbonates, phosphates, in particular alkalimetal phosphates, such as lithium, sodium or potassium phosphate. Suitable amine bases include but are not limited to tertiary organic amines, in particular aliphatic or cycloaliphatic tertiary amines, e.g. tri-C₁-C₄-alkylamines, C₃-C₆-cycloalkyl-di-C₁-C₄-alkylamines, tertiary cyclic amines and pyridines such as dimethylcyclohexylamine, trimethylamine, triethylamine, N-methylpiperidine, N-methylmorpholine, pyridine, 2,6-dimethylpyridine, 2,4,6-trimethylpyridine or quinoline. Preferred bases are alkalimetal carbonates, such as lithium, sodium or potassium carbonates and tertiary amines in particular triethylamine, pyridine, 2,6-dimethylpyridine or 2,4,6-trimethylpyridine.

In addition to or instead of the base, an amidation catalyst can be used. Suitable amidation catalysts are dialkylaminopyridines such as 4-(N,N-dimethylamino)pyridine (4-DMAP). The catalyst is usually employed in amounts from 0.001 to 1 mol, in particular from 0.005 to 0.2 mol, especially from 0.01 to 0.1 mol per mol of compound of formula (I).

In particular embodiments of the invention, the reaction of step (c) is carried out in an organic solvent or a mixture of organic solvents. Suitable solvents for carrying out the reaction of step (c) are preferably aprotic solvents and mixtures thereof. Examples of aprotic solvents are aliphatic hydrocarbons, such as alkanes, e.g. pentane, hexane or heptane, octane, cycloaliphatic hydrocarbons, such as cycloalkanes, e.g. cyclopentane or cyclohexane, halogenated alkanes, such as methylene chloride, chloroform or 1,2-dichlorethane, aromatic hydrocarbons, such as benzene, toluene, the xylenes, mesitylene or chlorobenzene, ester solvents, such as C₁-C₆-alkyl esters of an aliphatic C₁-C₄-carboxylic ester, in particular C₁-C₆-alkyl esters of acetic acid or propionic acid such as ethyl acetate, propyl acetate, butyl acetate or ethyl propionate, open-chained ethers, such as diethylether, methyl-tert-butyl ether, diisopropyl ether or methyl-isobutyl ether, cyclic ethers, such as tetrahydrofuran, 1,4-dioxane or 2-methyl tetrahydrofuran, nitriles, such as acetonitrile or propionitrile, the aforementioned pyridines such as pyridine, 2,6-dimethylpyridine or 2,4,6-trimethylpyridine, N,N-di-C₁-C₄-alkylamides of aliphatic carboxylic acids such as N,N-dimethylformamide, N,N-dimethylacetamide, and N—C₁-C₄-alkyl lactames such as N-methyl pyrrolidinone. Particular preferred solvents for carrying out reaction of step (c) are cyclohexane, dichloromethane, chlorobenzene, toluene, pyridine, tetrahydrofurane and N,N-dimethyl formamide, C₁-C₆-alkyl esters of an aliphatic C₁-C₄-carboxylic ester, in particular C₁-C₆-alkyl esters of acetic acid or propionic acid such as ethyl acetate, propyl acetate, butyl acetate or ethyl propionate, and mixtures thereof.

The reaction according to step (c) of the inventive process is generally performed at a temperature in the range of from −40 to +150° C., preferably from −10 to 110° C. and more preferably from 0 to 80° C. In principle the reaction temperature can be as high as the boiling point of the reaction mixture at the given reaction pressure, but is preferably kept at the indicated lower values. The reaction pressure is generally not critical and may range from 0.9 to 2 bar, in particular from 0.9 to 1.5 bar and especially from 0.9 to 1.1 bar.

The reaction of step (c) is carried out by reacting compound of formula (VII) with a suitable amount of a compound of formula (I) under the above reaction conditions. The reaction can be performed for example in the following manner: a solution or a suspension of the base and of the compound of formula (VII) in a suitable organic solvent is charged to a suitable reaction vessel. To this mixture, the compound of formula (I) is added, preferably as a solution or suspension in an organic solvent. Addition of the compound of formula (I) may be done as a single portion or preferably continuously or in several portions. To the resulting mixture, the catalyst may be added, if desired. The catalyst may be added either neat, in solution or as a suspension in a suitable organic solvent.

The compound of formula (VI) formed in reaction of step (c) can be isolated from the reaction mixture by customary methods, e.g. by removal of the base from the reaction mixture by either filtration or extraction with water, followed by concentration by distilling off the solvent. Alternatively, the reaction mixture can be diluted with water or diluted aqueous acids, like hydrochloric acid or dilute aqueous sulphuric acid, and cooled to a temperature between −30 and +30° C. to precipitate the amide compound from the solvent or solvent mixture. The precipitated amide compound VI can be separated from the liquid reaction mixture by conventional means, e.g. by filtration, centrifugation etc. The amide compound of formula (VI) can also be isolated from the reaction mixture by addition of water to the reaction mixture and extracting the thus obtained mixtures with a suitable solvent. Suitable solvents for extraction purposes are essentially immiscible with water and are capable of dissolving sufficient amounts of compound VI. It is also possible to concentrate the reaction mixture by distilling of the solvent, mixing the thus obtained residue with water and extracting the thus obtained mixture with a suitable solvent. Examples of suitable solvents are aliphatic hydrocarbons, such as alkanes, e.g. pentane, hexane or heptane, cycloaliphatic hydrocarbons, such as cycloalkanes, e.g. cyclopentane or cyclohexane, halogenated alkanes, such as methylene chloride or chloroform, aromatic hydrocarbons, such as benzene, toluene, the xylenes or chlorobenzene, open-chain ethers, such as diethylether, diisopropyl ether, di-n-propyl ether, di-n-butyl ether, methyl-tert-butyl ether, ethyl-tert.-butyl ether or methyl-isobutyl ether, or esters, in particular C₁-C₄ alkyl esters of acetic acid or propionic acid such as ethyl acetate, butyl acetate or ethyl propionate.

The thus obtained compound of formula (VI) can be further purified, e.g. by crystallization or by chromatography or combined measures. However, frequently, the product is already obtained in a purity which does not require further purification steps.

The invention relates to a process for preparing a compound of the formula (VII). This process is hereinafter termed “process VII”. According to a first embodiment, process VII comprises reacting a compound of the formula (VIII) with a compound of formula (IX), and according to a second embodiment, process VII comprises reacting a compound of the formula (VIII) with a compound of formula (X),

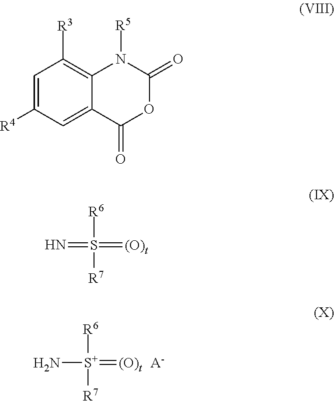

where R³, R⁴, R⁵, t, R⁶ and R⁷ are as defined herein and in the claims and where A⁻ is an equivalent of an anion having a pK_{B} of at least 10, as determined under standard conditions (298 K; 1.013 bar) in water.

For the conversion in process VII particular preference is given to compounds of the formula (VIII) wherein R⁵ is as defined herein and in the claims and where R³ has one of the meanings given herein and in the claims or is hydrogen, and R⁴ has one of the meanings given herein and in the claims or is hydrogen. Preferably, the radical R³ and R⁴ in formula (VIII) are, independently of each other, selected from the group consisting of hydrogen, halogen, C₁-C₄-alkyl, C₁-C₄-haloalkyl and cyano, it being possible that R³ and R⁴ are identical or different.

In the process VII of the present invention, preference is given to compounds of the formulae (IX) and (X), where the variable t is 0 and where R⁶ and R⁷, independently of each other, are selected from the group consisting of C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, wherein alkyl, alkenyl and cycloalkyl may optionally be substituted by one or more, e.g. 1 or 2 radicals R^{a}, where R^{a} is as defined above and in particular has one of the preferred meanings given above for R^{a}. Particular preference is given to compounds of the formulae (IX) and (X), where the variable t is 0 and where R¹ and R², independently of each other, are more preferably selected from the group consisting of C₁-C₆-alkyl, C₃-C₆-cycloalkyl and C₃-C₆-cycloalkyl-C₁-C₄-alkyl.

Likewise, preference is given to compounds of the formulae (IX) and (X), where the variable t is 0 and where R¹ and R² together represent a C₄-C₆-alkylene or C₄-C₆-alkenylene group forming together with the sulfur atom to which they are attached a 5-, 6- or 7-membered, saturated or partially unsaturated ring, wherein 1 or 2 of the CH₂ groups in the C₄-C₆-alkylene chain or 1 or 2 of any of the CH₂ or CH groups in the C₄-C₆-alkenylene chain may be replaced may be replaced by 1 or 2 groups independently selected from the group consisting of O, S, N and NH. Particular preference is also given to compounds of the formulae (IX) and (X), where the variable t is 0 and where R¹ and R² together preferably represent a C₄-C₆-alkylene group forming together with the sulfur atom to which they are attached a 5-, 6- or 7-membered saturated ring.

In the compounds of formula (X), A⁻ is an equivalent of an anion having a pK_{B} of at least 10, as determined under standard conditions (298 K; 1.013 bar) in water. In this context “equivalent” means the amount of anion required to achieve electroneutrality. For example, if the anion carries one negative charge the equivalent is 1, while if the anione carries two negative charges the equivalent is ½. Suitable anions are those, which have a basicity constant pK_{B} of at least 10, in particular at least 12 as determined under standard conditions (298 K; 1.013 bar) in water. Suitable anions include inorganic ions such as SO₄²⁻, HSO₄⁻, C₁⁻, ClO₄⁻, BF₄⁻, PF₆⁻, HPO₄⁻, and organic anions such as methylsulfonate, trifluoromethylsulfonate, trifluoroacetate, phenylsulfonate, toluenesulfonate, mesitylene sulfonate and the like.

In process VII, the compounds of formulae (IX) or (X), respectively, are typically employed in an amount of from 0.9 to 2 mol, preferably from 0.9 to 1.5 mol, more preferably from 0.9 to 1.2 mol and in particular from 0.95 to 1.1 mol per mol of the compound of formula (VIII) used in process VII.

It has been found advantageous to carry out the reaction of process VII in the presence of a base. Suitable bases include bases which are soluble or insoluble in the reaction medium. The base may be used in catalytic or stoichiometric amounts. The amount of base may preferably be in the range from 0.1 to 2 mol, in particular from 0.9 to 1.5 mol per mol of compound VIII or in the range from 0.1 to 2 mol, in particular from 0.9 to 1.5 mol per mol of compound IX or X. In a particular embodiment the base is used in an amount of at least 0.9 mol, in particular at least 1 mol, e.g. from 0.9 to 2 mol, in particular from 1 to 1.5 mol per mol of compound VIII, in particular, if a compound of formula (X) is used.

Suitable bases include but are not limited to oxo bases and amine bases. Suitable oxo bases include but are not limited to those mentioned in context with the reaction of scheme 1 herein before. Preferred bases are oxo bases, in particular alkalimetal alkoxides, which are also termed alkalimetal alkanolates, especially sodium and potassium alkanolates such as sodium methoxides, potassium methoxide, sodium ethoxide, potassium ethoxide, sodium tert-butanolate or potassium tert-butanolate. Mixtures of oxo bases and amine bases may also be used. Likewise preferred are bases which are selected from the aforementioned amine bases, in particular from the aforementioned tertiary amines.

In particular embodiments of the invention, the reaction of process VII is carried out in an organic solvent or a mixture of organic solvents. Suitable solvents for carrying out reaction VII may be protic or aprotic solvents and mixtures thereof, with aprotic solvents being preferred. Examples of aprotic solvents are aliphatic hydrocarbons, such as alkanes, e.g. pentane, hexane or heptane, cycloaliphatic hydrocarbons, such as cycloalkanes, e.g. cyclopentane or cyclohexane, halogenated alkanes, such as methylene chloride, chloroform or 1,2-dichlorethane, aromatic hydrocarbons, such as benzene, toluene, the xylenes or chlorobenzene, open-chain ethers, such as diethylether, methyl-tert-butyl ether, diisopropyl ether or methyl-isobutyl ether, cyclic ethers, such as tetrahydrofuran, 1,4-dioxane or 2-methyl tetrahydrofuran, esters, in particular the aforementioned C₁-C₄-alkyl acetates and propionates such as ethyl acetate, butyl acetate or ethyl propionate, aliphatic or alicyclic carbonates such as diethyl carbonate, ethylene carbonate (1,3-dioxolan-2-on) or propylene carbonate (4-methyl-1,2-dioxolan-2-on). Suitable aprotic solvents may also be pyridines such as pyridine, 2,6-dimethylpyridine or 2,4,6-trimethylpyridine, N,N-di-C₁-C₄-alkylamides of aliphatic carboxylic acids such as N,N-dimethyl formamide, N,N-dimethyl acetamide, and N—C₁-C₄-alkyl lactames such as N-methyl pyrrolidinone. Examples for polar protic solvents are C₁-C₄-alkanols, such as methanol, ethanol, propanol or isopropanol, C₂-C₄-alkandiols, such as ethylene glycol or propylene glycol, ether alkanols, such as diethylene glycol, sulfoxides, such as dimethyl sulfoxide, and mixtures thereof. Preferably the reaction is carried out in an aprotic solvent or a mixture of aprotic solvents.

The reaction according to process VII is generally performed at a temperature in the range of from −40 to +150° C., preferably from 0 to 110° C. and more preferably from 0 to 80° C. In principle the reaction temperature can be as high as the boiling point of the reaction mixture at the given reaction pressure, but is preferably kept at the indicated lower values. The reaction pressure is generally not critical and may range from 0.9 to 2 bar, in particular from 0.9 to 1.5 bar and especially from 0.9 to 1.1 bar.

The reaction of process VII is carried out by reacting compound VIII with a suitable amount of a compound of formulae (IX) or (X) under the above reaction conditions. The reaction can be performed for example in the following manner: a solution or a suspension of the compound of formula (VIII) in a suitable organic solvent is added to a suitable reaction vessel. To this mixture, the compound of formulae (IX) or (X) is added, preferably as a solution or suspension in an organic solvent. Addition of compound IX or X may be done as a single portion or preferably continuously or in several portions. To the resulting mixture, the base may be added, if desired. The base may be added either neat, in solution or as a suspension in a suitable organic solvent. Addition of the base may be done as a single portion or preferably continuously or in several portions. It is also possible to add the compound and, if desired, the base at the same time.

The compound of formula (VII) formed in reaction of process VII can be isolated from the reaction mixture by customary methods, e.g. by the addition of water and subsequent extraction with a suitable solvent, followed by concentration by distilling off the solvent. Suitable solvents for extraction purposes are essentially immiscible with water and capable of dissolving the compound of formula (VII). Examples are aliphatic hydrocarbons, such as alkanes, e.g. pentane, hexane or heptane, cycloaliphatic hydrocarbons, such as cycloalkanes, e.g. cyclopentane or cyclohexane, halogenated alkanes, such as methylene chloride or chloroform, aromatic hydrocarbons, such as benzene, toluene, the xylenes or chlorobenzene, open-chained ethers, such as diethylether, methyl-tert-butyl ether or methyl-isobutyl ether, or esters, such as ethyl acetate or ethyl propionate.

The isolated product can be further purified, e.g. by crystallization or by chromatography or combined measures. However, frequently, the product is already obtained in a purity which does not require further purification steps.

The compounds of formulae (IX) and (X) are known from prior art, e.g. from WO 2007/006670; WO 2008/141843; Y. Tamura et al, Tetrahedron 1975, 31, 3035-3040; Fujii et al., Heteroatom Chemistry 2004, 15(3), 246-250; Johnson et al., J. Org. Chem. 1989, 54, 986-988; Yoshimura et al., J. Org. Chem. 1976, 41, 1728-1733; Appel et al., Chem. Ber. 1962, 95, 849-854 and Chem. Ber. 1966, 99, 3108-3117; or from Young et al, J. Org. Chem. 1987, 52, 2695-2699; or they can be prepared by analogy to the methods described therein or by analogy to the methods described in WO 2008/141843, U.S. Pat. No. 6,136,983 and the literature cited therein.

The compounds of formula (VIII) are known from prior art, e.g. from WO 2003/016284 and Coppola, Synthesis 1980, pp. 505-536, or they can be prepared by analogy to the methods described therein. The compounds VIII can also be prepared by reacting an anthranilic acid derivative XIII with carbonic ester or an equivalent thereof such as phosgene, diphosgene (trichloromethyl chloroformiate), triphosgene (bis(trichloromethyl)carbonate), dialkyl carbonates, or alkyl chloroformiates as depicted in scheme 3.

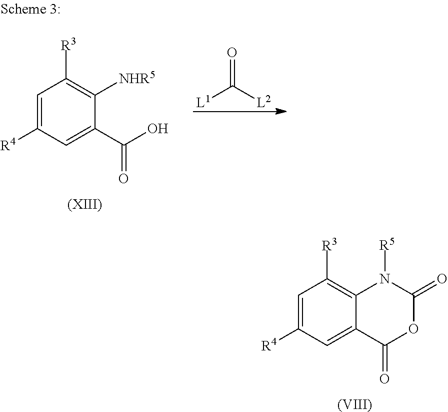

In scheme 3, R³, R⁴ and R⁵ are as defined above. L¹ is halogen, in particular chlorine, C₁-C₄-alkoxy, in particular methoxy or ethoxy, 1-imidazolyl or C₁-C₄-haloalkoxy such as trichloromethoxy. L² is halogen, in particular chlorine, trichloromethoxy, 1-imidazolyl, O—C(O)—Cl or C₁-C₄-alkoxy, in particular methoxy or ethoxy. Examples of suitable compounds of the formula C(O)L¹L² are phosgene, diphosgene, triphosgene, methyl or ethyl chloroformiate, carbonyldiimidazole, dimethylcarbonate and diethylcarbonate. The reaction of XIII with C(O)L¹L² can be achieved by analogy to the processes described in WO 2007/43677.

The reactions described herein are carried out in reaction vessels customary for such reactions, the reaction being configurable continuously, semicontinuously or batchwise.

### EXAMPLES

The compounds can be characterized e.g. by coupled Ultra High Performance Liquid Chromatography/mass spectrometry (HPLC/MS) and/or by coupled gas chromatography/mass spectrometry (GC/MS). The following analytical procedures were employed:

UHPLC/MS:

MSD4 and MSD5: Apparatus Shimadzu Nexera UHPLC: Binary pumpü LC-30AD, Autosampler SIL-30AC mit Rack Changer II, DAD SPD-M20A Shimadzu LCMS 20-20, ESI; Column: Phenomenex Kinetex 1.7 μm XB-C18 100A, 50×2.1 mm.
Mobile Phase: A: Water+0.1% trifluoroacetic acid (TFA); B: acetonitrile+0.1% TFA

### Temperature: 60° C.

Gradient: 5% B to 100% B within 1.50 min; 100% B 0.20 min
Flow: 0.8 ml/min to 1.0 ml/min within 1.50 min

### MS-Method: ESI-positive

Mass range (m/z): 100-700

### Analytical HPLC

### Column: Waters XBridge BEH130 C18 3,5μ 4.6*150 mm

Column Flow: 1.000 ml/min
Solvent A: 80% Water+25 g 0.05 mol/L H₂SO₄

### Solvent B: 20% Acetonitrile

### Timetable:

| Time | Solvent B | Flow | Pressure |
|---|---|---|---|
| 0.00 | 20 | 1.000 | 300 bar |
| 25.00 | 78 | 1.000 | 300 bar |
| 25.10 | 100 | 1.000 | 300 bar |
| 29.90 | 100 | 1.000 | 300 bar |
| 30.00 | 20 | 1.000 | 300 bar |

### Wavelength: 230 nm

Left temperature: 40.0° C.
Right temperature: 40.0° C.

GC/MS:

### Apparatus Agilent 6890 N/5975 B/MSD

### Column: Varian/50 m VF-1/ID 0.25 mm, FD 0.25 μm

Injector system: Agilent—Split Modus 1:20

### Injector: Agilent—Injektor 7683 B Serie/Menge=1 μl

### Detection: Agilent—MSD

### Injector: 270° C.

### MSD Interface: 280° C.

### Source: 230° C.

### MS Quad: 150° C.

Start time: t 12 min
Rate: 10°/min to 280° C.
Final time: 45 min

In the following examples and comparative examples the deprotonation of 3-chloro-2-[3-(trifluoromethyl)pyrazol-1-yl]pyridine is effected by methods of the present invention and the prior art, respectively. The level of deprotonation was determined via GC analysis subsequent to iodolysis which was performed as described below for each individual case. The contents of the final product, i.e. 2-(3-chloro-pyridin-2-yl)-5-(trifluoromethyl)pyrazole-3-carboxylic acid, in the obtained raw products was determined in each case by analytical HPLC.

### Example 1

### 2-(3-chloro-pyridin-2-yl)-5-(trifluoromethyl)pyrazole-3-carboxylic acid (by use of a lithium chloride complex of a magnesium amide of a secondary amine)

10.00 g (39.98 mmol, 1.00 eq) 3-chloro-2-[3-(trifluoromethyl)pyrazol-1-yl]pyridine (purity: 99%) were dissolved in 16 mL dry THF and the mixture was cooled down to 0° C. Then 46.11 g 2,2,6,6-tetramethylpiperidin-1-yl magnesium chloride lithium chloride complex (TMPMgCl.LiCl, 47.98 mmol, 1.20 eq) in THF (concentration: 25.22% by weight) was added within 3 hours at 0° C. After 45 min stirring at 0° C., an aliquot was taken and subjected to 0.5 mL solution of iodine in THF (0.5 g iodine in 1 mL THF). After 5 min the mixture was quenched with sodium thiosulfate and extracted with diethylether (1.5 mL). The GC analysis of the sample identified a ratio of 3-chloro-2-[3-(trifluoromethyl)pyrazol-1-yl]pyridine to 3-chloro-2-[5-iodo-3-(trifluoromethyl)pyrazol-1-yl]pyridine of 3.5 to 96.5. Subsequently carbon dioxide was bubbled into the vessel for 30 min at 0° C. and then 30 min at 20° C. An aliquot was taken, diluted with acetonitrile (MeCN) and analyzed by HPLC. The measured ratio of 3-chloro-2-[3-(trifluoromethyl)pyrazol-1-yl]pyridine to the title product was 4.2:95.8.

### Example 2

### 2-(3-chloro-pyridin-2-yl)-5-(trifluoromethyl)pyrazole-3-carboxylic acid (by use of a combination of a lithium chloride complex of a magnesium-organic compound having a carbon bound magnesium and a stoichiometric amount of a secondary amine)

9.23 g (50.38 mmol, 1.26 eq) of dicyclohexyl amine (purity: 99%) were added to a solution of 49.78 g (47.98 mmol, 1.20 eq) isopropyl magnesium chloride lithium chloride complex in THF (concentration: 14% by weight). The mixture was stirred for 1 h at 0° C. In a second vessel 10.00 g (39.98 mmol, 1.00 eq) 3-chloro-2-[3-(trifluoromethyl)pyrazol-1-yl]pyridine (purity: 99%) were dissolved in 9 mL dry THF and cooled down to 0° C. The in-situ prepared solution of dicyclohexylamido magnesium chloride lithium chloride complex was added within 3 hours to the second vessel and the mixture stirred for 3 h at 0° C. An aliquot was taken and subjected to 0.5 mL solution of iodine in THF (0.5 g iodine in 1 mL THF). After 5 min the mixture was quenched with aq. sodium thiosulfate and extracted with diethylether (1.5 mL). GC analysis of the sample identified a ratio of 3-chloro-2-[3-(trifluoromethyl)pyrazol-1-yl]pyridine to 3-chloro-2-[5-iodo-3-(trifluoromethyl)pyrazol-1-yl]pyridine of 3.5 to 96.5. Subsequently carbon dioxide was bubbled into the vessel for 30 min at 0° C. and 30 min at 20° C. An aliquot was taken, diluted with MeCN and analyzed by HPLC. The ratio of 3-chloro-2-[3-(trifluoromethyl)pyrazol-1-yl]pyridine to the title compound was 79.8:20.2.

### Example 3

### 2-(3-chloro-pyridin-2-yl)-5-(trifluoromethyl)pyrazole-3-carboxylic acid (by use of a combination of a lithium chloride complex of a magnesium-organic compound having a carbon bound magnesium and a catalytic amount of a secondary amine)

To 49.78 g (47.98 mmol, 1.20 eq) isopropyl magnesium chloride lithium chloride complex in THF (concentration: 14% by weight) was added 0.57 g (4.00 mmol, 0.10 eq) 2,2,6,6-tetramethylpiperidine (purity: 99%) and the mixture was stirred overnight at 0° C. In a second vessel 10.00 g (39.98 mmol, 1.00 eq) 3-chloro-2-[3-(trifluoromethyl)pyrazol-1-yl]pyridine (purity: 99%) were dissolved in 16 mL dry THF and cooled down to 0° C. The in-situ prepared solution of 2,2,6,6-tetramethylpiperidin-1-yl magnesium chloride lithium chloride complex was added within 3 hours to the second vessel and the mixture stirred for 4.5 h at 0° C. An aliquot was taken and subjected to 0.5 mL solution of iodine in THF (0.5 g iodine in 1 mL THF). After 5 min the mixture was quenched with aq. sodium thiosulfate and extracted with diethylether (1.5 mL). GC analysis of the sample identified a ratio of 3-chloro-2-[3-(trifluoromethyl)pyrazol-1-yl]pyridine to 3-chloro-2-[5-iodo-3-(trifluoromethyl)pyrazol-1-yl]pyridine of 9.9 to 90.1. Subsequently carbon dioxide was bubbled into the vessel for 30 min at 0° C. and 30 min at 20° C. An aliquot was taken, diluted with MeCN and analyzed by HPLC. The ratio of 3-chloro-2-[3-(trifluoromethyl)pyrazol-1-yl]pyridine to the title compound was 1.1:98.9.

### Example 4

### 2-(3-chloro-pyridin-2-yl)-5-(trifluoromethyl)pyrazole-3-carboxylic acid (by use of a combination of a lithium chloride complex of a magnesium-organic compound having a carbon bound magnesium and a catalytic amount of a secondary amine)

10.00 g (39.98 mmol, 1.00 eq) 3-chloro-2-[3-(trifluoromethyl)pyrazol-1-yl]pyridine (purity: 99%) and 0.41 g (4.00 mmol, 0.10 eq) diisopropyl amine (purity: 99%) were dissolved in 16 mL dry THF and cooled down to 0° C. Then 49.78 g (47.98 mmol, 1.20 eq) isopropyl magnesium chloride lithium chloride complex in THF (concentration: 14% by weight) was added within 3 hours. After further 3 hours stirring at 0° C., an aliquot was taken and subjected to 0.5 mL solution of iodine in THF (0.5 g iodine in 1 mL THF). After 5 min the mixture was quenched with aq. sodium thiosulfate and extracted with diethylether (1.5 mL). GC analysis of the sample identified a ratio of 3-chloro-2-[3-(trifluoromethyl)pyrazol-1-yl]pyridine to 3-chloro-2-[5-iodo-3-(trifluoromethyl)pyrazol-1-yl]pyridine of 7:93. Subsequently, carbon dioxide was bubbled into the vessel for 30 min at 0° C. and 30 min at 20° C. An aliquot was taken, diluted with MeCN and analyzed by HPLC. The ratio of 3-chloro-2-[3-(trifluoromethyl)pyrazol-1-yl]pyridine to the title compound was 4.7:95.3.

### Example 5

### 2-(3-chloro-pyridin-2-yl)-5-(trifluoromethyl)pyrazole-3-carboxylic acid (by use of a combination of a lithium chloride complex of a magnesium-organic compound having a carbon bound magnesium and a catalytic amount of a secondary amine)

10.00 g (39.98 mmol, 1.00 eq) 3-chloro-2-[3-(trifluoromethyl)pyrazol-1-yl]pyridine (purity: 99%) and 0.82 g (8.00 mmol, 0.20 eq) diisopropyl amine (purity: 99%) were dissolved in 16 mL dry THF and cooled down to 0° C. Then 49.78 g (47.98 mmol, 1.20 eq) isopropyl magnesium chloride lithium chloride complex in THF (concentration: 14% by weight) was added within 3 hours. After 1 hour stirring at 0° C., an aliquot was taken and subjected to 0.5 mL solution of iodine in THF (0.5 g iodine in 1 mL THF). After 5 min the mixture was quenched with aq. sodium thiosulfate and extracted with diethylether (1.5 mL). GC analysis of the sample identified a ratio of 3-chloro-2-[3-(trifluoromethyl)pyrazol-1-yl]pyridine to 3-chloro-2-[5-iodo-3-(trifluoromethyl)pyrazol-1-yl]pyridine of 3.2:96.8. Subsequently carbon dioxide was bubbled into the vessel for 30 min at 0° C. and further 30 min at 20° C. An aliquot was taken, diluted with MeCN and analyzed by HPLC. The ratio of 3-chloro-2-[3-(trifluoromethyl)pyrazol-1-yl]pyridine to the title compound was 10.3:89.7. Then the reaction mixture was cooled down to 0° C. and 35 mL water were added dropwise resulting in a mixture of pH 8. The pH was brought to 4 by adding about 5 g of concentrated hydrochloric acid. The mixture was extracted with 35 mL ethyl acetate and the separated aqueous phase was extracted again with 20 mL ethyl acetate. The combined organic phases were dried over sodium sulfate, filtered and concentrated on a rotary evaporator to yield 12.7 g of the title compound (52% purity, as determined by analytical HPLC, 26.66 mmol, yield: 67%).

### Comparative Example 1

### 2-(3-chloro-pyridin-2-yl)-5-(trifluoromethyl)pyrazole-3-carboxylic acid (by use of a lithium chloride complex of a magnesium-organic compound having a carbon bound magnesium alone)

10.00 g (39.98 mmol, 1.00 eq) 3-chloro-2-[3-(trifluoromethyl)pyrazol-1-yl]pyridine (purity: 99%) were dissolved in 16 mL dry THF and cooled down to 0° C. Then 49.78 g (47.98 mmol, 1.20 eq) isopropyl magnesium chloride lithium chloride complex in THF (concentration: 14% by weight) was added within 3 hours at −1° C. to 0° C. After 16 hours stirring at 0° C., an aliquot was taken and subjected to 0.5 mL solution of iodine in THF (0.5 g iodine in 1 mL THF). After 5 min the mixture was quenched with aq. sodium thiosulfate and extracted with diethylether (1.5 mL). GC analysis of the sample identified a ratio of 3-chloro-2-[3-(trifluoromethyl)pyrazol-1-yl]pyridine to 3-chloro-2-[5-iodo-3-(trifluoromethyl)pyrazol-1-yl]pyridine of 7.5 to 92.7. Subsequently carbon dioxide was bubbled into the vessel for 30 min at 0° C. and further 30 min at 20° C. An aliquot was taken, diluted with MeCN and analyzed by HPLC. The ratio of 3-chloro-2-[3-(trifluoromethyl)pyrazol-1-yl]pyridine to the title compound was 2.1:97.9.

### Comparative Example 2

### Deprotonation of 3-chloro-2-[3-(trifluoromethyl)pyrazol-1-yl]pyridine by use of a lithium-diisopropylamide

10.00 g (39.98 mmol, 1.00 eq) 3-chloro-2-[3-(trifluoromethyl)pyrazol-1-yl]pyridine (purity: 99%) were dissolved in 16 mL dry THF and the mixture was cooled down to 0° C. Then a solution of 32.47 g (79.97 mmol, 2.00 eq) lithium-diisopropylamide in THF/heptane/ethylbenzene (concentration: 26.38% by weight) was added within 3 hours and the resulting mixture was stirred for 1 h at 0° C. An aliquot was taken and subjected to 0.5 mL solution of iodine in THF (0.5 g iodine in 1 mL THF). After 5 min the mixture was quenched with aq. sodium thiosulfate and extracted with diethylether (1.5 mL). GC analysis of the sample identified a ratio of 3-chloro-2-[3-(trifluoromethyl)pyrazol-1-yl]pyridine to 3-chloro-2-[5-iodo-3-(trifluoromethyl)pyrazol-1-yl]pyridine of 92.4 to 7.6.

### Comparative Example 3

### Deprotonation of 3-chloro-2-[3-(trifluoromethyl)pyrazol-1-yl]pyridine by use of a lithium-diisopropylamide

4.72 g (46.18 mmol, 1.16 eq) of diisopropyl amine (purity: 99%) were dissolved in 5.5 mL dry THF and cooled down to 0° C. Then a solution of 18.69 g (43.98 mmol, 1.10 eq) n-butylithium in hexane (concentration: 15.07% by weight) was added within 5 min and the mixture was stirred for 1 h at 0° C. In a second vessel 10.00 g (39.98 mmol, 1.00 eq) 3-chloro-2-[3-trifluoromethyl)pyrazol-1-yl]pyridine (purity: 99%) was dissolved in 10.5 mL dry THF and cooled down to 0° C. The in-situ prepared solution of lithium-diisopropylamide was added within 3 hours. The mixture was stirred for 1 h at 0° C. An aliquot was taken and subjected to 0.5 mL solution of iodine in THF (0.5 g iodine in 1 mL THF). After 5 min the mixture was quenched with aq. sodium thiosulfate and extracted with diethylether (1.5 mL). The sample was analyzed by GC. Neither 3-chloro-2-[3-(trifluoromethyl)pyrazol-1-yl]pyrid ne nor 3-chloro-2-[5-iodo-3-(trifluoromethyl)pyrazol-1-yl]pyridine could be detected. Also after carboxylation with carbon dioxide the expected educt and product could not be detected by HPLC.

The results obtained in the Examples performed in accordance to the present invention are summarized in the following table. The Examples 1 to 5 are carried out as described above. The Examples 6 to 8, 11 and 12 are carried out in analogy to the procedure of Example 4, the example 9 is carried out by analogy to example 3 and the Example 10 is carried out in analogy to the procedure of Example 2. The “E:I ratio” and “E:P ratio” given in the table represents the ratio of 3-chloro-2-[3-(trifluoromethyl)pyrazol-1-yl]pyridine to 3-chloro-2-[5-iodo-3-(trifluoromethyl)pyrazol-1-yl]pyridine and of 3-chloro-2-[3-(trifluoromethyl)pyrazol-1-yl]pyridine to 2-(3-chloro-pyridin-2-yl)-5-(trifluoromethyl)pyrazole-3-carboxylic acid, respectively, as determined from the peak areas of the respective GC and HPLC diagrams obtained as described in the Examples.

| | Mg amide or secondary amine | | |
|---|---|---|---|
| Example | used, equivalents | E:I ratio | E:P ratio |
| Ex. 1 | TMPMgCl•LiCl, 1.20 eq | 3.5:96.5 | 4.2:95.8 |
| Ex. 2 | dicyclohexylamine, 1.26 eq | 3.5:96.5 | 79.8:20.2 |
| Ex. 6 | dicyclohexylamine, 0.20 eq | 1.9:98.1 | 9.2:90.8 |
| Ex. 7 | dicyclohexylamine, 0.10 eq | 4.3:95.7 | 6.6:93.4 |
| Ex. 3 | TMP, 0.10 eq | 9.9:90.1 | 1.1:98.9 |
| Ex. 4 | diisopropylamine, 0.10 eq | 7.0:93.0 | 4.7:95.3 |
| Ex. 5 | diisopropylamine, 0.20 eq | 3.2:96.8 | 10.3:89.7 |
| Ex. 8 | dimethylamine, 0.10 eq | 17.0:82.0 | 2.6:97.4 |
| Ex. 9 | N-tert-butyl-N-isopropyl amine, | 9.4:90.6 | 5.7:94.3 |
| | 0.20 eq | | |
| Ex. 10 | N,N-bis(2-ethylhexyl) amine, | 9.4:90.6 | 87.1:12.9 |
| | 1.26 eq | | |
| Ex. 11 | N,N-bis(2-ethylhexyl) amine, | 2.2:97.8 | 5.1:94.9 |
| | 0.20 eq | | |
| Ex. 12 | N,N-bis(2-ethylhexyl), amine, | 6.2:93.8 | 5.5:94.5 |
| | 0.10 eq | | |

TMPMgCl•LiCl: 2,2,6,6-tetramethylpiperidin-1-yl magnesium chloride lithium chloride complex
TMP: 2,2,6,6-tetramethylpiperidin

### Example 13

### 2-(3-chloro-pyridin-2-yl)-5-(trifluoromethyl)pyrazole-3-carboxylic acid (by use of a combination of a lithium chloride complex of a magnesium-organic compound having a carbon bound magnesium and a catalytic amount of a secondary amine)

10.00 g (40.27 mmol, 1.00 eq) 3-chloro-2-[3-(trifluoromethyl)pyrazol-1-yl]pyridine (purity: 99.7%) and 0.59 g (8.00 mmol, 0.20 eq)N-methyl-N-isopropyl amine (purity: 99%) were dissolved in 14.5 g dry THF and cooled down to 0° C. Then 50.13 g (42.8 mmol, 1.06 eq) of a 12.4% b.w. solution of isopropyl magnesium chloride lithium chloride complex in THF was added within 3 hours. After further 3 hours stirring at 0° C., an aliquot was taken and subjected to 0.5 mL solution of iodine in THF (0.5 g iodine in 1 mL THF). After 5 min the mixture was quenched with aq. sodium thiosulfate and extracted with diethylether (1.5 mL). GC analysis of the sample identified a ratio of 3-chloro-2-[3-(trifluoromethyl)pyrazol-1-yl]pyrid ne to 3-chloro-2-[5-iodo-3-(trifluoromethyl)pyrazol-1-yl]pyridine of 3:97. Subsequently, carbon dioxide was bubbled into the vessel for 30 min at −2° C. and 30 min at 20° C. An aliquot was taken, diluted with acetonitrile and analyzed by HPLC. The ratio of 3-chloro-2-[3-(trifluoromethyl)pyrazol-1-yl]pyridine to the title compound was 1:99.

### Example 14

### 2-(3-chloro-pyridin-2-yl)-5-(trifluoromethyl)pyrazole-3-carboxylic acid (by use of a combination of lithium chloride, magnesium-organic compound having a carbon bound magnesium and a catalytic amount of a secondary amine)

126.5 g 3-chloro-2-[3-(trifluoromethyl)pyrazol-1-yl]pyridine (purity: 94.6%) were dissolved in 70 g dry THF. 10.4 g of lithium chloride were added followed by addition of 1.8 g of N-isopropyl-N-methyl amine. The mixture was cooled to −10° C. Then 287 g of a 19.1% b.w. solution of isopropyl magnesium chloride in THF was added within 1.2 h at −10° C. to −2° C. The obtained reaction mixture was stirred for further 3.5 h at 0 to −10° C. An aliquot was taken and subjected to 0.5 mL solution of iodine in THF (1 g iodine in 2 mL THF). After 5 min the mixture was quenched with aq. sodium thiosulfate and extracted with diethylether (20 mL). GC analysis of the sample identified a ratio of 3-chloro-2-[3-(trifluoromethyl)pyrazol-1-yl]pyridine to 3-chloro-2-[5-iodo-3-(trifluoromethyl)pyrazol-1-yl]pyridine of 3.5:96.5. Subsequently, carbon dioxide was bubbled into the vessel for 95 min at −16° C. to 2° C. An aliquot was taken, added to aqueous 10% b.w. of hydrochloric acid, diluted with water and extracted with dichloromethane. The extract was analyzed by HPLC. The ratio of 3-chloro-2-[3-(trifluoromethyl)pyrazol-1-yl]pyridine to the title compound was 0.6:99.4. For work up, the reaction mixture was diluted with THF to a total of 500 ml. The mixture was concentrated in vacuo and the distilled solvent was replaced several times by butyl acetate until about 500 g of a suspension containing 158.4 g of the MgCl-Salt of the title compound in butyl acetate was obtained. This corresponds to a yield of 93.6%.

1-15. (canceled)

16. A process for preparing an N-substituted 1H-pyrazole-5-carboxylic acid of the formula (I-A) or a magnesium salt thereof 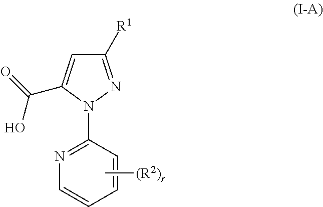
wherein
R¹ is selected from hydrogen, fluorine, chlorine, cyano, —SF₅, CBrF₂, C₁-C₆-alkyl, C₁-C₆-fluoroalkyl, C₃-C₈-cycloalkyl, C₃-C₈-fluorocycloalkyl, C₂-C₆-alkenyl, C₂-C₆-fluoroalkenyl, wherein the six last mentioned radicals may be substituted by one or more radicals R^{a};
—Si(R^{f})₂R^{g}, —OR^{b}, —SR^{b}, —S(O)ₘR^{b}, —S(O)ₙN(R^{c})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R^{e};
each R² is independently selected from the group consisting of halogen, SF₅, C₁-C₆-alkyl, C₁-C₆-fluoroalkyl, C₃-C₈-cycloalkyl, C₃-C₈-fluorocycloalkyl, C₂-C₆-alkenyl, C₂-C₆-fluoroalkenyl, wherein the six last mentioned radicals may be substituted by one or more radicals R^{a};
—Si(R^{f})₂R^{g}, —OR^{b}, —SR^{b}, —S(O)ₘR^{b}, —S(O)ₙN(R^{c})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e};
R^{a} is selected from the group consisting of SF₅, C₁-C₆-alkyl, C₁-C₆-fluoroalkyl, C₁-C₆-alkoxy-C₁-C₆-alkyl, C₃-C₈-cycloalkyl, C₃-C₈-fluorocycloalkyl, C₂-C₆-alkenyl, C₂-C₆-fluoroalkenyl, —Si(R^{f})₂R^{g}, —OR^{b}, —SR^{b}, —S(O)ₘR^{b},
—S(O)ₙN(R^{c})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e};
or two geminally bound radicals R^{a} together form a group selected from ═CR^{h}Rⁱ, ═NR^{c1}, ═NOR^{b} and ═NNR^{c1},
or two radicals R^{a}, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;
wherein, in the case of more than one R^{a}, R^{a} can be identical or different;
R^{b} is selected from the group consisting of C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, wherein the six last mentioned radicals may optionally carry 1 or 2 radicals selected from C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-fluoroalkylsulfinyl, C₁-C₆-alkylsulfonyl, C₁-C₆-fluoroalkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;
wherein, in the case of more than one R^{b}, R^{b} can be identical or different;
R^{c}, R^{d} are, independently from one another and independently of each occurrence, selected from the group consisting of cyano, C₁-C₆-fluoroalkyl, C₂-C₆-alkenyl, C₂-C₆-fluoroalkenyl, C₃-C₈-cycloalkyl, C₃-C₈-fluorocycloalkyl, wherein the six last mentioned radicals may optionally carry 1 or 2 radicals selected from C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-alkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;
or R^{c} and R^{d}, together with the nitrogen atom to which they are bound, form a 3-, 4-, 5-, 6- or 7-membered saturated, partly unsaturated or completely unsaturated heterocyclic ring which may contain 1 or 2 further heteroatoms selected from N, O and S as ring members, where the heterocyclic ring may carry 1, 2, 3 or 4 substituents selected from halogen, C₁-C₄-alkyl, C₁-C₄-fluoroalkyl, C₁-C₄-alkoxy and C₁-C₄-fluoroalkoxy;
R^{c1} is hydrogen or has one of the meanings given for R^{c};
R^{d1} is hydrogen or has one of the meanings given for R^{d};
R^{e} is selected from the group consisting of halogen, C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, where the six last mentioned radicals may optionally carry 1 or 2 radicals selected from C₁-C₄-alkoxy; C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-fluoroalkylsulfinyl, C₁-C₆-alkylsulfonyl, C₁-C₆-fluoroalkylsulfonyl,
—Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;
wherein, in the case of more than one R^{e}, R^{e} can be identical or different;
R^{f}, R^{g} are, independently of each other and independently of each occurrence, selected from the group consisting of C₁-C₄-alkyl, C₃-C₆-cycloalkyl, C₁-C₄-alkoxy-C₁-C₄-alkyl, phenyl and benzyl;
R^{h}, Rⁱ are, independently from one another and independently of each occurrence, selected from the group consisting of hydrogen, halogen, SF₅, C₁-C₆-fluoroalkyl, C₂-C₆-alkenyl, C₂-C₆-fluoroalkenyl, C₃-C₈-cycloalkyl, C₃-C₈-fluorocycloalkyl, where the six last mentioned radicals may optionally carry 1 or 2 radicals selected from C₁-C₄-alkyl and C₁-C₄-fluoroalkyl; C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-alkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy, C₁-C₆-fluoroalkoxy, (C₁-C₆-alkoxy)carbonyl, (C₁-C₆-alkyl)amino and di-(C₁-C₆-alkyl)amino;
m is 1 or 2, wherein, in the case of several occurrences, m may be identical or different;
n is 0, 1 or 2; wherein, in the case of several occurrences, n may be identical or different;
r is 0, 1, 2, 3 or 4;
which process comprises the following steps:
i) reacting a compound of the formula (II) 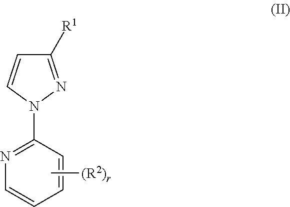
in which the variables R¹, R² and r are each as defined above,
with a base selected from (a) a combination of a magnesium-organic compound having a carbon bound magnesium and a secondary amine and (b) a magnesium amide of a secondary amine in the presence of a lithium halide,
where the base is used in an amount sufficient to achieve at least 80% deprotonation of the compound of formula (II); and
ii) subjecting the product obtained in step (i) to a carboxylation by reacting it with carbon dioxide or a carbon dioxide equivalent, to obtain a magnesium salt of the compound of formula (I-A) and optionally aqueous workup to obtain the compound of the formula (I-A) as a free acid.

17. The process of claim 16, wherein the secondary amine is selected from
di-C₁-C₁₄-alkyl amines,
N—C₁-C₁₄-alkyl-N-cycloalkyl amines
di-C₃-C₈-cycloalkyl amines,
saturated 5- to 7-membered heterocyclic amines, which are unsubstituted or substituted by 1, 2, 3 or 4 C₁-C₈-alkyl groups, where the heterocycle in addition to the NH group may contain 1 or 2 further heteroatoms selected from O and N, which are in the form of an ether oxygen or in the form of a N—C₁-C₄-alkyl group, and
N-aryl-N—C₁-C₁₄-alkyl amines where the aryl group is unsubstituted or substituted by 1, 2, 3 or 4 radicals selected from C₁-C₄-alkyl and C₁-C₄-alkoxy.

18. The process of claim 16, wherein the secondary amine is a compound of the formula (RN) 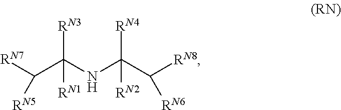
wherein R^{N1}, R^{N2}, R^{N3}, R^{N4}, R^{N5}, R^{N6}, R^{N7}, R^{N8}, independently of each other are selected from the group consisting of hydrogen and C₁-C₈-alkyl, where the two radicals R^{N3} and R^{N4} may also form a (CH₂)ₚ group with p being 2, 3 or 4, wherein one CH₂ group may be replaced by an oxygen atom or a group N—C₁-C₄-alkyl;
provided that the total number of carbon atom in the radicals R^{N1}, R^{N2}, R^{N3}, R^{N4}, R^{N5}, R^{N6}, R^{N7} and R^{N8} is from 2 to 24.

19. The process of claim 18, wherein in formula (RN)
R^{N1} and R^{N2} are hydrogen or methyl, R^{N3} and R^{N4} are C₁-C₄-alkyl or form a (CH₂)ₚ group with p being 2, 3 or 4, wherein one CH₂ group in (CH₂)ₚ may be replaced by an oxygen atom or a group N—C₁-C₄-alkyl and R^{N5}, R^{N6}, R^{N7} and R^{N8} are hydrogen, or R^{N1} and R^{N2} are hydrogen or methyl, R^{N3} and R^{N4} are hydrogen and R^{N5}, R^{N6}, R^{N7} and R^{N8} are C₁-C₈-alkyl.

20. The process of claim 16, wherein the secondary amine is selected from the group consisting of dimethylamine, diethylamine, dicyclohexylamine, piperidine, morpholine, 2,2,6,6-tetramethylpiperidine, 2,2,4,6,6-pentamethylpiperazine, 3,3,5,5-tetramethylmorpholine, N-methyl-N-isopropylamine, N-ethyl-N-isopropylamine, N,N-diisopropylamine, N-isopropyl-N-tert-butylamine, N,N-di-tert-butylamine, N,N-bis-(2-ethylhexyl)amine, N,N-bis-(2-propylheptyl)amine, N,N-bis-(2-butyloctyl)amine and N-methyl-N-phenylamine.

21. The process of claim 16, wherein the amount of secondary amine is from 0.01 mol to 2 mol per 1 mol of the compound of formula (II).

22. The process of claim 16, wherein the amount of the base, calculated as magnesium, is from 1 mol to 2 mol per 1 mol of the compound of formula (II).

23. The process of claim 16, wherein the base is a combination of a magnesium organic compound selected from the group consisting of C₁-C₆-alkyl magnesium halides and C₅-C₆-cycloalkyl magnesium halides.

24. The process of claim 16, wherein the lithium halide is lithium chloride.

25. The process of claim 16, where the lithium halide is used in an amount from 0.5 to 2 mol per mol of magnesium in the base.

26. The process of claim 16, wherein steps i) and ii) are performed in an aprotic organic solvent or aprotic organic solvent mixture comprising at least one aprotic organic solvent having an ether moiety.

27. The process of claim 16, where in formulae (I-A) and (II)
r is 1, and
R² is located in position 3 of the pyridyl moiety and where R² is selected from halogen and CF₃;
R¹ is selected from the group consisting of fluorine, chlorine, C₁-C₄-fluoroalkyl, C₁-C₄-alkoxy and C₁-C₄-fluoroalkoxy-C₁-C₄-alkyl.

28. A process for preparing an N-substituted 1H-pyrazole-5-carbonyl chloride of the formula (I): 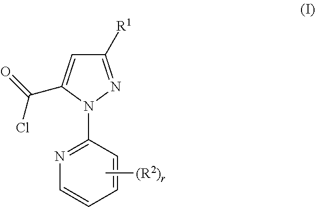
wherein
R¹ is selected from hydrogen, fluorine, chlorine, cyano, —SF₅, CBrF₂, C₁-C₆-alkyl, C₁-C₆-fluoroalkyl, C₃-C₈-cycloalkyl, C₃-C₈-fluorocycloalkyl, C₂-C₆-alkenyl, C₆-fluoroalkenyl, wherein the six last mentioned radicals may be substituted by one or more radicals R^{a};
—Si(R^{f})₂R^{g}, —OR^{b}, —SR^{b}, —S(O)ₘR^{b}, —S(O)ₙN(R^{c})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R^{e};
each R² is independently selected from the group consisting of halogen, SF₅, C₁-C₆-alkyl, C₁-C₆-fluoroalkyl, C₃-C₈-cycloalkyl, C₃-C₈-fluorocycloalkyl, C₂-C₆-alkenyl, C₂-C₆-fluoroalkenyl, wherein the six last mentioned radicals may be substituted by one or more radicals R^{a};
Si(R^{f})₂R^{g}, —OR^{b}, —SR^{b}, —S(O)ₘR^{b}, —S(O)ₙN(R^{c})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e};
R^{a} is selected from the group consisting of SF₅, C₁-C₆-alkyl, C₁-C₆-fluoroalkyl, C₁-C₆-alkoxy-C₁-C₆-alkyl, C₃-C₈-cycloalkyl, C₃-C₈-fluorocycloalkyl, C₂-C₆-alkenyl, C₂-C₆-fluoroalkenyl, —Si(R^{f})₂R^{g}, —OR^{b}, —SR^{b}, —S(O)ₘR^{b},
—S(O)ₙN(R^{c})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e};
or two geminally bound radicals R^{a} together form a group selected from ═CR^{h}Rⁱ, ═NR^{c1}, ═NOR^{b} and ═NNR^{c1},
or two radicals R^{a}, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;
wherein, in the case of more than one R^{a}, R^{a} can be identical or different;
R^{b} is selected from the group consisting of C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, wherein the six last mentioned radicals may optionally carry 1 or 2 radicals selected from C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-fluoroalkylsulfinyl, C₁-C₆-alkylsulfonyl, C₁-C₆-fluoroalkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;
wherein, in the case of more than one R^{b}, R^{b} can be identical or different;
R^{c}, R^{d} are, independently from one another and independently of each occurrence, selected from the group consisting of cyano, C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, wherein the six last mentioned radicals may optionally carry 1 or 2 radicals selected from C₁-C₆-alkoxy, C₁-C₆-fluoroalkoxy, C₁-C₆-alkylthio, C₁-C₆-fluoroalkylthio, C₁-C₆-alkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;
or R^{c} and R^{d}, together with the nitrogen atom to which they are bound, form a 3-, 4-, 5-, 6- or 7-membered saturated, partly unsaturated or completely unsaturated heterocyclic ring which may contain 1 or 2 further heteroatoms selected from N, O and S as ring members, where the heterocyclic ring may carry 1, 2, 3 or 4 substituents selected from halogen, C₁-C₄-alkyl, C₁-C₄-fluoroalkyl, C₁-C₄-alkoxy and C₁-C₄-fluoroalkoxy;
R^{c1} is hydrogen or has one of the meanings given for R^{e};
R^{d1} is hydrogen or has one of the, meanings given for R^{d};
R^{e} is selected from the group consisting of halogen, C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, where the six last mentioned radicals may optionally carry 1 or 2 radicals selected from C₁-C₄-alkoxy; C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-fluoroalkylsulfinyl, C₁-C₆-alkylsulfonyl, C₁-C₆-fluoroalkylsulfonyl,
—Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;
wherein, in the case of more than one R^{e}, R^{e} can be identical or different;
R^{f}, R^{g} are, independently of each other and independently of each occurrence, selected from the group consisting of C₁-C₄-alkyl, C₃-C₆-cycloalkyl, C₁-C₄-alkoxy-C₁-C₄-alkyl, phenyl and benzyl;
R^{h}, Rⁱ are, independently from one another and independently of each occurrence, selected from the group consisting of hydrogen, halogen, SF₅, C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, where the six last mentioned radicals may optionally carry 1 or 2 radicals selected from C₁-C₄-alkyl and C₁-C₄-fluoroalkyl; C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-alkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy, C₁-C₆-fluoroalkoxy, (C₁-C₆-alkoxy)carbonyl, (C₁-C₆-alkyl)amino and di-(C₁-C₆-alkyl)amino;
m is 1 or 2, wherein, in the case of several occurrences, m may be identical or different;
n is 0, 1 or 2; wherein, in the case of several occurrences, n may be identical or different;
r is 0, 1, 2, 3 or 4;
which comprises
a) preparing an N-substituted 1H-pyrazole-5-carboxylic acid of the formula (I-A) or a magnesium salt thereof by a process of claim 16; and
b) converting the compound of the formula (I-A) or the magnesium salt into the compound of formula (I) by treatment with a chlorinating agent.

29. The process of claim 28, where in step a) the magnesium salt of the compound of formula (I-A) is prepared which is directly converted to the compound of formula (I) by treatment with a chlorinating agent.

30. A process for preparing a sulfimine compound of formula (VI) 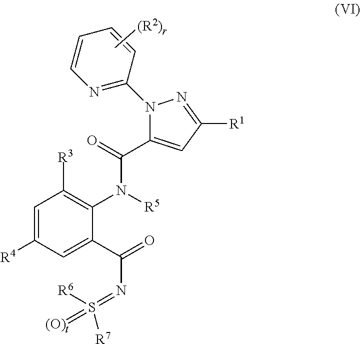
in which
R¹ is selected from hydrogen, fluorine, chlorine, cyano, —SF₅, CBrF₂, C₁-C₆-alkyl, C₁-C₆-fluoroalkyl, C₃-C₈-cycloalkyl, C₃-C₈-fluorocycloalkyl, C₂-C₆-alkenyl, C₂-C₆-fluoroalkenyl, wherein the six last mentioned radicals may be substituted by one or more radicals R^{a};
—Si(R^{f})₂R^{g}, —OR^{b}, —SR^{b}, —S(O)ₘR^{b}, —S(O)ₙN(R^{c})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R^{e};
each R² is independently selected from the group consisting of halogen, SF₅, C₁-C₆-alkyl, C₁-C₆-fluoroalkyl, C₃-C₈-cycloalkyl, C₃-C₈-fluorocycloalkyl, C₂-C₆-alkenyl, C₂-C₆-fluoroalkenyl, wherein the six last mentioned radicals may be substituted by one or more radicals R^{a};
—Si(R^{f})₂R^{g}, —OR^{b}, —SR^{b}, —S(O)—N(R^{c})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e};
R^{a} is selected from the group consisting of SF₅, C₁-C₆-alkyl, C₁-C₆-fluoroalkyl, C₁-C₆-alkoxy-C₁-C₆-alkyl, C₃-C₈-cycloalkyl, C₃-C₈-fluorocycloalkyl, C₂-C₆-alkenyl, C₂-C₆-fluoroalkenyl, —Si(R^{f})₂R^{g}, —OR^{b}, —SR^{b}, —S(O)ₘR^{b},
—S(O)ₙN(R^{c})R^{d}, —N(R^{c1})R^{d1}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e};
or two geminally bound radicals R^{a} together form a group selected from ═CR^{h}Rⁱ, ═NR^{c1}, ═NOR^{b} and ═NNR^{c1},
or two radicals R^{a}, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;
wherein, in the case of more than one R^{a}, R^{a} can be identical or different;
R^{b} is selected from the group consisting of C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, wherein the six last mentioned radicals may optionally carry 1 or 2 radicals selected from C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-fluoroalkylsulfinyl, C₁-C₆-alkylsulfonyl, C₁-C₆-fluoroalkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;
wherein, in the case of more than one R^{b}, R^{b} can be identical or different;
R^{c}, R^{d} are, independently from one another and independently of each occurrence, selected from the group consisting of cyano, C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, wherein the six last mentioned radicals may optionally carry 1 or 2 radicals selected from C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-alkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;
or R^{c} and R^{d}, together with the nitrogen atom to which they are bound, form a 3-, 4-, 5-, 6- or 7-membered saturated, partly unsaturated or completely unsaturated heterocyclic ring which may contain 1 or 2 further heteroatoms selected from N, O and S as ring members, where the heterocyclic ring may carry 1, 2, 3 or 4 substituents selected from halogen, C₁-C₄-alkyl, C₁-C₄-fluoroalkyl, C₁-C₄-alkoxy and C₁-C₄-fluoroalkoxy;
R^{c1} is hydrogen or has one of the meanings given for R^{c};
R^{d1} is hydrogen or has one of the meanings given for R^{d};
R^{e} is selected from the group consisting of halogen, C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, where the six last mentioned radicals may optionally carry 1 or 2 radicals selected from C₁-C₄-alkoxy; C₁-C₆-alkoxy, C₁-C₆-fluoroalkoxy, C₁-C₆-alkylthio, C₁-C₆-fluoroalkylthio, C₁-C₆-fluoroalkylsulfinyl, C₁-C₆-alkylsulfonyl, C₁-C₆-fluoroalkylsulfonyl,
—Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy and C₁-C₆-fluoroalkoxy;
wherein, in the case of more than one R^{e}, R^{e} can be identical or different;
R^{f}, R^{g} are, independently of each other and independently of each occurrence, selected from the group consisting of C₁-C₄-alkyl, C₃-C₆-cycloalkyl, C₁-C₄-alkoxy-C₁-C₄-alkyl, phenyl and benzyl;
R^{h}, Rⁱ are, independently from one another and independently of each occurrence, selected from the group consisting of hydrogen, halogen, SF₅, C₁-C₆-alkyl, C₁-C₆-fluoroalkyl, C₂-C₆-alkenyl, C₂-C₆-fluoroalkenyl, C₃-C₈-cycloalkyl, C₃-C₈-fluorocycloalkyl, where the six last mentioned radicals may optionally carry 1 or 2 radicals selected from C₁-C₄-alkyl and C₁-C₄-fluoroalkyl; C₁-C₆-alkoxy, C₁-C₆-fluoroalkoxy, C₁-C₆-alkylthio, C₁-C₆-fluoroalkylthio, C₁-C₆-alkylsulfinyl, C₁-C₆-alkylsulfonyl, —Si(R^{f})₂R^{g}, phenyl, benzyl, pyridyl and phenoxy, wherein the four last mentioned radicals may be unsubstituted, partially or fully halogenated and/or may carry 1, 2 or 3 substituents selected from the group consisting of C₁-C₆-alkyl, C₁-C₆-fluoroalkyl, C₁-C₆-alkoxy, C₁-C₆-fluoroalkoxy, (C₁-C₆-alkoxy)carbonyl, (C₁-C₆-alkyl)amino and di-(C₁-C₆-alkyl)amino;
m is 1 or 2, wherein, in the case of several occurrences, m may be identical or different;
n is 0, 1 or 2; wherein, in the case of several occurrences, n may be identical or different;
r is 0, 1, 2, 3 or 4;
R³ and R⁴ are independently selected from the group consisting of halogen, cyano, azido, nitro, —SCN, SF₅, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the eight last mentioned radicals may be substituted by one or more radicals R^{a}; —Si(R^{f})₂R^{g}, —OR^{b1}, —OS(O)ₙR^{b1}, —SR^{b1}, —S(O)ₘR^{b1}, —S(O)—N(R^{c1})R^{d1}, —N(R^{c1})R^{d1}, —N(R^{c1})C(═O)R^{a}, —C(═O)R^{a}, —C(═O)OR^{b1}, —C(═S)R^{a}, —C(═S)OR^{b1}, —C(═NR^{c1})R^{a}, —C(═O)N(R^{c1})R^{d1}, —C(═S)N(R^{c1})R^{d1}, phenyl which may be substituted by 1, 2, 3, 4 or 5 radicals R^{c}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{c};
R⁵ is selected from the group consisting of hydrogen, cyano, C₁-C₁₀-alkyl, C₃-C₈-cycloalkyl, C₃-C₈-halocycloalkyl, C₂-C₁₀-alkenyl, C₂-C₁₀-haloalkenyl, C₂-C₁₀-alkynyl, C₂-C₁₀-haloalkynyl, wherein the eight last radicals may optionally be substituted by one or more radicals R^{a}; —N(R^{c1})R^{d1}, —Si(R^{f})₂R^{g}, —OR^{b1}, —SR^{b1}, —S(O)ₘR^{b1}, —S(O)ₙN(R^{c1})R^{d1}, C(═O)R^{a}, —C(═O)OR^{b1}, C(═O)N(R^{c1})R^{d1}, —C(═S)R^{a}, —C(═S)OR^{b1}, —C(═S)N(R^{c1})R^{d1}, —C(═NR^{c1})R^{a}, phenyl which may be substituted by 1, 2, 3, 4 or 5 radicals R^{e}, and a 3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members, where the heterocyclic ring may be substituted by one or more radicals R^{e};
R⁶ and R⁷ are selected independently of one another from the group consisting of hydrogen, C₁-C₁₀-alkyl, C₁-C₁₀-haloalkyl, C₃-C₈-cycloalkyl, C₃-C₈-halocycloalkyl, C₂-C₁₀-alkenyl, C₂-C₁₀-haloalkenyl, C₂-C₁₀-alkynyl, C₂-C₁₀-haloalkynyl, wherein the eight last radicals may optionally be substituted by one or more radicals R^{a};
or R⁶ and R⁷ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or completely unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy,
C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl; said substituents being identical or different from one another if more than one substituent is present;
R^{a}, R^{c1}, R^{d1}, R^{e}, R^{f}, R^{g}, m and n are each as defined in claim 16;
R^{b1} is hydrogen or has one of the meanings given for R^{b} in claim 16; and
t is 0 or 1;
which comprises providing a compound of the formula (I) 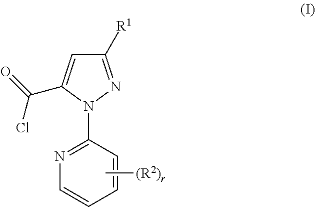
and
c) reacting the compound of the formula (I) with a compound of the formula (VII) 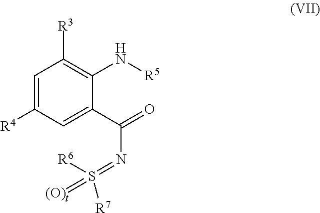
in which the variables R³, R⁴, R⁵, R⁶, R⁷ and t are each as defined above,
in the presence of a base, to obtain a compound of the formula (VI).
